(19) United States
(12) Patent Application Publication
(10) Pub. No.: US 2022/0175691 A1
(43) Pub. Date: Jun. 9, 2022

(54) DELIVERY SYSTEMS FOR RELEASE OF ACTIVE COMPOUNDS

(71) Applicant: Textile-Based Delivery, Inc., Conover, NC (US)

(72) Inventors: David Anderson, Rockford, MN (US); Jordan Schindler, Tuscon, AZ (US); Erik Scott Goebel, Vadnais Heights, MN (US); Nathan Jablonske, Hickory, NC (US); Sandy Wood, East Bend, NC (US); Michael Drzewinski, Long Valley, NJ (US); Justin Jed, Seattle, WA (US)

(73) Assignee: Textile-Based Delivery, Inc., Conover, NC (US)

(21) Appl. No.: 17/678,612

(22) Filed: Feb. 23, 2022

Related U.S. Application Data
(63) Continuation-in-part of application No. 17/007,962, filed on Aug. 31, 2020.
Application No. 17/007,962 is a continuation of application No. 16/216,581, filed on Dec. 11, 2018, now Pat. No. 10,799,464.
Application No. 16/216,581 is a continuation-in-part of application No. 15/996,126, filed on Jun. 1, 2018.
Application No. 15/996,126 is a continuation of application No. 15/583,584, filed on May 1, 2017, now abandoned.
Application No. 15/583,584 is a continuation of application No. 14/926,949, filed on Oct. 29, 2015, now Pat. No. 9,669,012.
(60) Provisional application No. 62/072,896, filed on Oct. 30, 2014.

(51) Int. Cl. A61K 9/70; A61K 31/343; A61K 31/573; A61K 31/555; A61K 31/203; A61K 31/122; A61K 31/12; A61K 31/4412; A61K 9/00; A61L 15/24; A61L 15/40; A61L 15/44; A61K 31/4166; A61K 31/616; A61K 47/34; A61M 37/00

(52) U.S. Cl. CPC A61K 9/70; A61L 2300/43; A61K 31/573; A61K 31/555; A61K 31/203; A61K 31/122; A61K 31/12; A61K 31/4412; A61K 9/0024; A61K 9/0092; A61L 15/24; A61L 15/40; A61L 15/44; A61K 31/4166; A61K 31/616; A61K 47/34; A61M 37/00; A61L 2300/402; A61L 2300/41; A61K 31/343

(57) ABSTRACT

Drug delivery systems and wearable articles including the drug delivery systems are provided. The drug delivery systems may include a substrate coated with at least one polymer and at least one active compound. The substrate is operable to include yarns, yarn precursors, threads, filaments, fibers, and/or other suitable substrates. Methods for manufacturing drug delivery systems are also provided. The methods are operable to include disposing a solution including a monomer and an active compound on the substrate. The methods are also operable to include exposing the solution and the substrate to UV light to initiate polymerization of the solution.

### CROSS REFERENCES TO RELATED APPLICATIONS

This application is related to and claims priority from the following U.S. patents and patent applications. This application is a continuation-in-part of U.S. patent application Ser. No. 17/007,962, filed Aug. 31, 2020, which is a continuation of U.S. patent application Ser. No. 16/216,581, which issued as U.S. Pat. No. 10,799,464, which is a continuation-in-part of U.S. patent application Ser. No. 15/996,126, filed Jun. 1, 2018, which is a continuation of U.S. patent application Ser. No. 15/583,584, filed May 1, 2017, which is a continuation of U.S. patent application Ser. No. 14/926,949, filed Oct. 29, 2015, which issued as U.S. Pat. No. 9,669,012, which claims the benefit of and priority to U.S. Provisional Patent Application No. 62/072,896, filed Oct. 30, 2014. Each of the above listed applications are incorporated herein by reference in its entirety.

### BACKGROUND OF THE INVENTION

### 1. Field of the Invention

The present disclosure relates to materials configured to deliver a variety of active compounds via coated and uncoated yarns and other substrates, as well as methods to produce such yarns or substrates. The present disclosure further relates to methods for making and manufacturing drug delivery systems.

### 2. Description of the Prior Art

It is generally known in the prior art to provide substrates including at least one active component.

Prior art patent documents include the following:

U.S. Patent Publication No. 20170281561 for Drug delivery fabric having drug-containing layer by inventors Go, et al., filed Aug. 28, 2014 and published Oct. 5, 2017, is directed to a drug delivery fabric having a drug-containing layer, wherein the drug delivery fabric comprises a surface layer, a middle layer, and a drug-containing layer, the middle layer is formed of a yarn having hydrophilicity relatively equal to or higher than that of the surface layer, the drug-containing layer is formed of a cellulose derivative film containing a drug.

U.S. Patent Publication No. 20080242174 for Wash resistant synthetic polymer compositions containing active compounds by inventor Sun, filed Mar. 26, 2008 and published Oct. 2, 2008, is directed to a synthetic polymer composition comprising a blend of a synthetic thermoplastic polymer and a polymer containing a polyether chain as a constituent, wherein the article manufactured from the blend contains an active compound. Yarns may be formed from the synthetic polymer blend and then knitted or woven into articles, such as net or fabric. The articles may be treated with active compounds such as a perfume, fabric softener, sunscreen agent, antibacterial agent, pesticide, insecticide and such other compounds that provide functionality on the article. The treated articles retain more than an effective amount of active compounds even after numerous washings.

U.S. Pat. No. 8,241,921 for Active agent eluting matrices with particulates by inventors Slager, et al., filed Dec. 28, 2009 and issued Aug. 14, 2012, is directed to polymeric matrices for the controlled release of a hydrophilic bioactive agent. Generally, the elution control matrix includes a polymeric matrix having a first polymer and a plurality of microparticles that include the hydrophilic bioactive agent. The matrix includes a polymer comprising hydrophilic and hydrophobic portions. Alternatively, the microparticles include a crosslinked hydrophilic polymer.

U.S. Pat. No. 6,291,371 for application of film forming technology to fragrance control release systems; and resultant fragrance control release system by inventors Shefer, et al., filed Jul. 21, 1999 and issued Sep. 18, 2001, is directed to a fragrance control release system which is an emulsifier-free, single phase, nonporous, continuous, permeable polymeric film having a substantially uniform thickness of from about 1 up to about 150 microns, having entrapped and dissolved therein molecules of at least one fragrance substance capable of evolving from said film into the environment proximate said film by means of molecular diffusion at a permeation rate of from about 1×10-7 up to about 0.1 mg-mm/cm2-min in a sustained and controlled release manner. Also described is a process for using the aforementioned system for imparting a fragrance into the environment above the unobstructed outer surface of the aforementioned polymer film which is coated on the surface of a solid or semi-solid support, e.g., a woven or non-woven fabric substrate; or a solid surface or human epidermis.

U.S. Pat. No. 5,788,687 for Compositions and devices for controlled release of active ingredients by inventors Batich, et al., filed Jul. 31, 1995 and issued Aug. 4, 1998, is directed to a method for the controlled release of a biologically active agent wherein the agent is released from a hydrophobic, pH-sensitive polymer matrix. The patent discloses that the polymer matrix swells when the environment reaches pH 8.5, releasing the active agent. A polymer of hydrophobic and weakly acidic comonomers is disclosed for use in the controlled release system. The patent also discloses that weakly basic comonomers are used and the active agent is released as the pH drops. The patent further discloses that the pH-sensitive polymer is coated onto a latex catheter used in ureteral catheterization. A common problem with catheterized patients is the infection of the urinary tract with urease-producing bacteria. In addition to the irritation caused by the presence of the bacteria, urease produced by these bacteria degrade urea in the urine, forming carbon dioxide and ammonia. The ammonia causes an increase in the pH of the urine. Minerals in the urine begin to precipitate at this high pH, forming encrustations which complicate the functioning of the catheter. A ureteral catheter coated with a pH-sensitive polymer having an antibiotic or urease inhibitor trapped within its matrix releases the active agent when exposed to the high pH urine as the polymer gel swells. Such release can be made slow enough so that the drug remains at significant levels for a clinically useful period of time. Other uses for the methods and devices include use in gastrointestinal tubes, respiratory trap lines and ventilation tubes, dye releasing pH sensitive sutures, active agent release from contact lenses, penile implants, heart pacemakers, neural shunts, food wraps, and clean room walls.

U.S. Pat. No. 7,119,060 for Controlled delivery system for fabric care products by inventors Shefer, et al., filed Jan. 15, 2003 and issued Oct. 10, 2006, is directed to a controlled delivery system that can be incorporated in liquid, as well as, dry granular, or powder, fabric care products, such as fabric softeners, laundry detergents, rinse added products, and other fabric care products, to enhance fragrance performance. The controlled delivery system is a solid, substantially spherical particle comprising hydrophobic cationic charge enhancing agents in conjunction with cationic fabric softening agents that assist in adhering the particles onto fabric. The particles can also include a fragrance. The particle can have an average particle diameter of from about 1 micron to about 500 microns. The controlled delivery system can be utilized to deliver a broad range of fragrance ingredients onto fabric and prolong fragrance release from the dry laundered fabric over an extended period of time, or yield a high impact fragrance “burst” upon ironing the fabric. The patent also pertains to fabric care products comprising the controlled release system.

U.S. Pat. No. 5,232,769 for Microcapsule, treating liquids containing the same, and textile structure having microcapsules adhering thereto by inventors Yamato, et al., filed Jul. 31, 1990 and issued Aug. 3, 1993, is directed to a microcapsule having a particle diameter of 2^{˜}300 μm and comprising a substance acting to improve physiological conditions of human skin, for example, substances exhibiting such effects as skin whitening, aging preventive, humidity preservable, itch suppressive, pain-killing, or antiphlogistic ones, and/or aromatic agents contained within the filmy coating of synthetic high molecular substance. The microcapsule is not broken when making, processing, or laundering the textile structure, but is gradually broken when the textile structure is put on the human body, used for another purpose, or subjected to intentional application of friction or pressure thereto, and sustainedly releases acting substances contained therein. Treatment liquids comprising these microcapsules and binder, preferably containing a spraying agent, adapt the microcapsules to tightly adhere to textile structures such as stockings underwear, and bedclothes, thereby providing a textile structure to exhibit the aforesaid effects.

U.S. Pat. No. 5,869,172 for Internally-coated porous webs with controlled positioning of modifiers therein by inventor Caldwell, filed May 17, 1995 and issued Feb. 9, 1999, is directed to processes for treating a porous substrate (especially a fabric) to produce novel internally coated porous materials. During treatment, a curable thixotropic material and one or modifying materials are applied to the porous substrate as an impregnant. The treatment imparts specific properties to the end product material. Selection of the modifier material is based on the particular end use application. Sufficient energy is directed to the impregnant and porous substrate to cause the impregnant to flow into the porous substrate and force the modifier to specific positions within the substrate.

U.S. Patent Publication No. 20050033251 for Controlled release of biologically active substances from select substrates by inventors Toreki, et al., filed Feb. 25, 2004 and published Feb. 10, 2005, is directed to methods and compositions for materials having a non-leaching coating that has antimicrobial properties. The coating is applied to substrates such as gauze-type wound dressings, powders and other substrates. Covalent, non-leaching, non-hydrolyzable bonds are formed between the substrate and the polymer molecules that form the coating. A high concentration of anti-microbial groups on multi-length polymer chains and relatively long average chain lengths, contribute to an absorbent or superabsorbent surface with a high level antimicrobial efficacy. Utilization of non-leaching coatings having a plurality of anionic or cationic sites is used to bind a plurality of oppositely charged biologically or chemically active compounds, and to release the bound oppositely charged biologically or chemically active compounds from said substrate over a period of time to achieve desired objectives as diverse as improved wound healing to reduction in body odor.

### SUMMARY OF THE INVENTION

The present disclosure relates to materials configured to deliver a variety of active compounds via coated and uncoated yarns and other substrates, as well as methods to produce such yarns or substrates. The present disclosure further relates to methods for making and manufacturing drug delivery systems.

In one embodiment, the present invention provides a wearable article including a fabric formed with at least one yarn, yarn precursor, or filament having a polymer, oligomer, or monomer matrix including at least one active composition applied thereto, wherein the at least one yarn, yarn precursor, or filament includes at least one occluded area and at least one exposed area, wherein the at least one occluded area restricts release of the at least one active composition, and wherein the at least one exposed area does not restrict release of the at least one active composition, and wherein the wearable article persistently releases the at least one active composition when in contact with a skin surface of a wearer via the at least one exposed area.

In another embodiment, the present invention provides a wearable article including a knitted structure formed with at least one substrate having a polymer, oligomer, or monomer matrix including at least one active composition applied thereto, wherein the knitted structure includes at least one occluded area and at least one exposed area, wherein the at least one occluded area restricts release of the at least one active composition, and wherein the at least one exposed area does not restrict release of the at least one active composition, wherein the knitted structure is operable to substantially conform to at least one body part of a wearer, and wherein the wearable article persistently releases the at least one active composition when in contact with a skin surface of a wearer via the at least one exposed area.

In yet another embodiment, the present invention provides a wearable article including a fabric formed with at least one substrate having a polymer, oligomer, or monomer matrix including at least one active composition applied thereto, wherein the at least one substrate includes at least one occluded area and at least one exposed area, wherein the at least one occluded area restricts release of the at least one active composition, and wherein the at least one exposed area does not restrict release of the at least one active composition, wherein the wearable article is operable to substantially conform to at least one body part of a wearer, wherein the at least one body part is a finger, a hand, a wrist, an elbow, an arm, a shoulder, a torso, a head, a face, a neck, a toe, a foot, an ankle, a knee, a hip, and/or a leg, and wherein the wearable article persistently releases the at least one active composition when in contact with a skin surface of a wearer via the at least one exposed area.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1 is a perspective view of an embodiment of an intermittently coated yarn including a single type of coating.

FIG. 2 is a perspective view of an embodiment of an intermittently coated yarn including two types of coatings.

FIG. 3 is a perspective view of an embodiment of a yarn including an outer sheath or coating.

FIG. 4 is a graph of ultraviolet (UV) absorbance versus the square root of time in days for certain samples of active compounds, according to an embodiment of the present disclosure.

FIG. 5 is a photograph of a thin layer chromatography plate spotted with samples of active compounds, according to an embodiment of the present disclosure.

FIG. 6 is a graph of UV absorbance data showing the near-zero order release of an active compound, usnic acid, according to an embodiment of the present disclosure.

FIG. 7 is a graph of UV absorbance data showing the near-zero order release of an active compound, terbinafine hydrochloride, according to an embodiment of the present disclosure.

FIG. 8 is a graph of visible light absorbance data showing the near-zero order release of an active compound, dantrolene, according to an embodiment of the present disclosure.

FIG. 9 is a photograph depicting a coated yarn, according to an embodiment of the present disclosure.

FIG. 10 is a photograph depicting a coated yarn, according to another embodiment of the present disclosure.

FIG. 11 is a graph showing active retention after laundering.

FIG. 12 is a graph showing WS-23 release from yarn.

FIG. 13A is a graph showing nonivamide release from various polymeric coatings.

FIG. 13B is a table listing the various polymeric coatings in FIG. 13A.

FIG. 14 is a graph showing active remaining in washed samples.

FIG. 15A is a graph showing extraction of nonivamide from sleeves loaded with 3% nonivamide.

FIG. 15B is a graph showing extraction of nonivamide from sleeves loaded with 5% nonivamide.

### DETAILED DESCRIPTION

The present disclosure relates to materials configured to deliver a variety of active compounds via coated and uncoated yarns and other substrates, as well as methods to produce such yarns or substrates. In one embodiment, the materials are configured to exhibit zero-order or near-zero-order release of the active compounds. Additionally or alternatively, the materials are also configured to protect the active compounds from loss so as to provide a therapeutic amount of active even after repeated uses, wears, applications, and/or launderings. In one embodiment, the materials are also configured to provide substantive protection against hydrolysis and other forms of degradation.

The present disclosure further relates to methods for making and manufacturing drug delivery systems. In certain embodiments, the drug delivery systems include a substrate coated or covered with at least one polymer and at least one active compound. In particular embodiments, the at least one polymer that coats the substrate is an acrylate polymer or a methacrylate polymer. In specific embodiments, the at least one polymer is cross-linked. In further embodiments, the at least one polymer is a hydrophobic polymer. The substrate includes yarns, yarn precursors, threads, filaments, fibers, textiles, and/or other suitable substrates. In one embodiment, the methods include applying or disposing a mixture or solution including at least one polymer, oligomer, or monomer and at least one active compound onto the substrate. In one embodiment, the methods include subjecting or exposing the mixture or solution on the substrate to ultraviolet (UV) light to initiate polymerization and/or cross-linking of the solution.

In one embodiment, the present invention provides a wearable article including a fabric formed with at least one yarn, yarn precursor, or filament having a polymer, oligomer, or monomer matrix including at least one active composition applied thereto, wherein the at least one yarn, yarn precursor, or filament includes at least one occluded area and at least one exposed area, wherein the at least one occluded area restricts release of the at least one active composition, and wherein the at least one exposed area does not restrict release of the at least one active composition, and wherein the wearable article persistently releases the at least one active composition when in contact with a skin surface of a wearer via the at least one exposed area. In one embodiment, the wearable article is an ankle sleeve, an arm sleeve, a calf sleeve, a knee sleeve, a lower leg sleeve, a wrist sleeve, a sock, a glove, leggings, partial leggings, pants, partial pants, a shirt, or a partial shirt. In one embodiment, the at least one active composition includes a capsaicinoid. In one embodiment, the capsaicinoid is nonivamide. In one embodiment, the at least one active composition is persistently released following at least one wash when the wearable article is in contact with the skin surface of the wearer. In one embodiment, the wearable article is comprised of a knitted fabric formed from the at least one yarn, yarn precursor, or filament. In one embodiment, a structural integrity and the persistent release of the at least one composition are maintained after at least one wash, and the wearable article is reusable after at least one wash. In one embodiment, the wearable article is operable to substantially conform to at least one body part of a wearer. In one embodiment, the wearable article is seamless. In one embodiment, the wearable article loses less than about 25% of the at least one active composition present in the material during a wash cycle. In one embodiment, the at least one occluded area comprises at least 80% of a surface area of the at least one yarn, yarn precursor, or filament. In one embodiment, the fabric further includes an air-covered yarn. In one embodiment, the wearable article produces a compressive force when worn. In one embodiment, the at least one occluded area is formed using at least one coating, and wherein the at least one coating is substantially impermeable to the at least one active composition.

In another embodiment, the present invention provides a wearable article including a knitted structure formed with at least one substrate having a polymer, oligomer, or monomer matrix including at least one active composition applied thereto, wherein the knitted structure includes at least one occluded area and at least one exposed area, wherein the at least one occluded area restricts release of the at least one active composition, and wherein the at least one exposed area does not restrict release of the at least one active composition, wherein the knitted structure is operable to substantially conform to at least one body part of a wearer, and wherein the wearable article persistently releases the at least one active composition when in contact with a skin surface of a wearer via the at least one exposed area. In one embodiment, the wearable article is an ankle sleeve, an arm sleeve, a calf sleeve, a knee sleeve, a lower leg sleeve, a wrist sleeve, a sock, a glove, leggings, partial leggings, pants, partial pants, a shirt, or a partial shirt. In one embodiment, the knitted structure is formed with a first substrate and a second substrate, wherein the first substrate includes the at least one active composition, and wherein the second substrate does not include an active composition. In one embodiment, the at least one active composition includes a first active composition and a second active composition, wherein the at least one substrate includes a first substrate and a second substrate, wherein the first substrate includes the first active composition, and wherein the second substrate includes the second active composition. In one embodiment, the at least one substrate is a yarn, a yarn precursor, a thread, a fiber, a filament, or a textile.

In yet another embodiment, the present invention provides a wearable article including a fabric formed with at least one substrate having a polymer, oligomer, or monomer matrix including at least one active composition applied thereto, wherein the at least one substrate includes at least one occluded area and at least one exposed area, wherein the at least one occluded area restricts release of the at least one active composition, and wherein the at least one exposed area does not restrict release of the at least one active composition, wherein the wearable article is operable to substantially conform to at least one body part of a wearer, wherein the at least one body part is a finger, a hand, a wrist, an elbow, an arm, a shoulder, a torso, a head, a face, a neck, a toe, a foot, an ankle, a knee, a hip, and/or a leg, and wherein the wearable article persistently releases the at least one active composition when in contact with a skin surface of a wearer via the at least one exposed area.

Prior art systems are generally operable to release at least one active composition (e.g., transdermal patches), but are unable to be washed or reused. Wearable articles (e.g., socks) must often be washed between uses for hygienic reasons. Additionally, many prior art systems are designed to be antimicrobial. However, these antimicrobial systems are often designed using superabsorbent polymers and, thus, are not washable, or are designed to trap particles instead of release an active composition. Other prior art systems require incorporating the at least one active composition in a polymer matrix to form a synthetic fiber containing the at least one active composition. However, this limits the materials to synthetic fibers, which may be undesirable. Additionally, this prevents use of materials further down the supply chain and complicates the manufacturing process. Existing coating of pre-made garments is an external treatment that alters the overall feel, texture, and stiffness of a garment, and also creates a system that does not withstand repetitive washings.

In contrast, the present invention provides a wearable article or garment that is operable to release at least one active composition. In one embodiment, the wearable article or garment is formed using yarns, yarn precursors, threads, filaments, fibers, textiles, and/or substrates that are operable to persistently release the at least one active composition. Advantageously, the wearable article or garment and/or the yarns, yarn precursors, threads, filaments, fibers, textiles, and/or substrates are operable to persistently release the at least one active composition after at least one wash cycle. In one embodiment, the yarns, yarn precursors, threads, filaments, fibers, textiles, and/or substrates form a knitted structure. Advantageously, the knitted structure is operable to provide both a relaxed state and a stretched state. In one embodiment, the wearable article or garment is formed of the knitted structure. Alternatively, the yarns, yarn precursors, threads, filaments, fibers, textiles, and/or substrates form a woven structure. In one embodiment, the yarns, yarn precursors, threads, filaments, fibers, textiles, and/or substrates are operable to be used for cut and sew. The wearable article or garment contacts at least one body part of a wearer (e.g., patch, tape, wrap, etc.). In a preferred embodiment, the wearable article or garment is operable to substantially conform to the at least one body part of a wearer. The at least one body part includes, but is not limited to, a finger, a hand, a wrist, an elbow, an arm, a shoulder, a torso, a head, a face, a neck, a toe, a foot, an ankle, a knee, a hip, and/or a leg. In one embodiment, the wearable article or garment is seamless. Advantageously, a seamless wearable article or garment provides additional comfort to a wearer by reducing points of friction. In an alternative embodiment, the wearable article or garment is formed of a woven textile. The present invention is compatible with yarns, yarn precursors, threads, filaments, fibers, textiles, and/or substrates formed of natural and/or synthetic materials.

None of the prior art discloses wearable articles, wearable garments, and/or yarns, yarn precursors, threads, filaments, fibers, textiles, and/or substrates that are operable to release at least one active composition after laundering. There is a long-felt, unmet need for wearable articles, wearable garments, and/or yarns, yarn precursors, threads, filaments, fibers, textiles, and/or substrates that are operable to persistently release at least one active composition even after laundering. Further, there is a long-felt, unmet need for wearable articles, wearable garments, and/or yarns, yarn precursors, threads, filaments, fibers, textiles, and/or substrates including natural fibers (e.g., cotton) that are treated with at least one active composition.

Referring now to the drawings in general, the illustrations are for the purpose of describing one or more preferred embodiments of the invention and are not intended to limit the invention thereto.

The present disclosure provides yarns, yarn precursors, threads, fibers, filaments, textiles, and/or other substrates (e.g., films, sheets, patches, cut and sew pieces) that are loaded with biologically active compounds, compositions, or ingredients (also referred to herein as “actives” and/or “active particles”) that are integrated into the yarns, yarn precursors, threads, fibers, filaments, textiles, and/or substrates. These delivery systems are operable to be utilized to release the active compounds onto or into mammalian tissue, including, for example, human skin.

As used herein, the terms “yarn” and “yarn precursor” include not only finished yarns, but also starting or intermediate fiber based materials from, e.g., greige cotton or extruded filament, to finished and as described in certain embodiments, functionalized yarns (e.g., yarns that are loaded with an active compound), whether on, e.g., a cone or spool or in a textile or fabric. The term “yarn” can also be used to describe individual threads and spun and/or twisted threads. In some embodiments, the yarn is bulked or textured. Bulked and/or textured yarns can refer to yarns that have been treated mechanically, chemically, or physically (e.g., tension-adjusted) so as to appear to have greater or increased volume relative to the yarn prior to mechanical, chemical, or physical treatment. For example, bulked and/or textured yarns are operable to have a crimped, coiled, or spiral configuration rather than a linear or stretched configuration. Bulked and/or textured yarns are operable to exhibit favorable properties over, e.g., partially-oriented yarn (POY) or other yarns lacking texture and/or bulk.

A number of advantages accompany the maintenance of bulk or texture in yarns loaded with active compounds as disclosed herein including, but not limited to, comfort, compatibility with established textile production, and/or high surface area in the non-occluded segments of the yarn. One factor to maintaining texture is selecting the coating and the matrix polymer such that they rapidly skin-over upon application. In some embodiments, this is achieved by applying solvent-free (e.g., water-free) matrix polymers and coatings, as aqueous dispersions (often denoted “latex” coatings or paints) may not readily yield a textured or bulked final yarn upon application to a textured or bulked precursor, unless strong conditions are used to flash off the water in a very short time (e.g., one second or less).

Embodiments of this disclosure provide yarns, yarn precursors, threads, filaments, fibers, fabrics, and other textiles, and other substrates that release therapeutically effective amounts of active compounds (e.g., organic active compounds) to the skin of a mammal. Such active compounds are operable to be selected for their dermatological and/or cosmetic benefit, e.g., for skin health and beauty. The active compounds are operable to penetrate into the skin or be delivered to tissue below the skin, including to the bloodstream. In certain embodiments, the active compound(s) are operable to penetrate into or through the skin to a depth that depends on the active concentration, the yarn-to-skin (or substrate-to-skin) contact time, physicochemical properties of the active, and/or the structure and condition of the skin.

Embodiments of this disclosure also provide yarns, yarn precursors, threads, filaments, fibers, textiles, and/or other substrates that release a therapeutically effective amount of active compound into the bloodstream of a mammal from outside the body. For instance, in one embodiment, this includes transdermal delivery, wherein contact of the yarn, the yarn precursor, the thread, the filament, the fiber, the textile, or the substrate with mammalian skin results in transfer of one or more active compounds through the skin and into the bloodstream. Textiles, fabrics, clothing, or apparel comprising yarns, yarn precursors, threads, filaments, fibers, and/or other substrates that deliver or release therapeutic amounts of active compounds to, or through, the skin of a mammal that makes contact with the textile, the fabric, the clothing, or the apparel are also provided.

Embodiments of this disclosure also provide yarns, yarn precursors, threads, filaments, fibers, textiles, fabrics, and/or substrates that are able to withstand washing and other stresses physical, chemical, thermal, weather) with minimal or no loss of active. Thus, the present invention provides cold washable and hot-washable yarns, yarn precursors, threads, filaments, fibers, textiles, fabrics, and/or substrates that are loaded with active. For example, in a normal washing machine hot wash cycle, these yarns, yarn precursors, threads, filaments, fibers, textiles, fabrics, and/or substrates are operable to lose less than about 23%, less than about 12%, less than about 7%, less than about 3%, or less than about 1% of the active that was present in the material just before the wash.

The embodiments of the present disclosure include individual yarns, yarn precursors, threads, filaments, fibers, and other substrates, which are operable to provide flexibility through the blending of various active-loaded yarns, yarn precursors, threads, filaments, fibers, and other substrates. Advantageously, the embodiments of the present disclosure further provide low shipping costs to overseas mills and markets, especially as compared to finished fabrics (because the medicated yarn need only be a small fraction of the overall fabric yarn). Furthermore, the present invention provides the ability to provide the consumer with medicated thread that is operable to be applied to a fabric with a household sewing machine. The present invention further provides the opportunity to produce a product that is earlier farther upstream in the value-added chain that spans from raw fiber to finished textile.

Furthermore, the various embodiments of the present disclosure are operable to include or utilize cross-linked, hydrophobic polymers (e.g., elastomers such as silicone, rubbers and fluoroelastomers) as protective matrices for actives. Cross-linking (also referred to as “curing,” “vulcanizing,” and “thermosetting”) applied to a dispersion or suspension of active particles in a polymer, oligomer, or monomer matrix such as a Room Temperature Vulcanizer (RTV), commercial coating or adhesive, chemically reactive linear polymer, etc. is operable to be employed by the various embodiments of the present disclosure for preparing yarns, yarn precursors, threads, filaments, fibers, textiles, fabrics, or substrates to protect the active against excessive loss during laundering, as well as against a wide range of chemical degradation reactions including hydrolysis, oxidation (depending on the polymer), acid/base-catalyzed reactions, etc. The polymer matrices are operable to be formed from various polymer- or oligomer-based systems, including commercially available elastomeric adhesives, glues, coatings, caulks, sealants, casting materials, and cross-linking systems. The polymers (e.g., elastomers) are also operable to be formed from one or more monomers.

In specific embodiments, the polymers (e.g., elastomers) are used as a vehicle to load one or more actives into and/or onto the yarn, yarn precursor, thread, filament, fiber, textile, or other substrate and/or immobilize the one or more actives in and/or on the yarn, yarn precursor, thread, filament, fiber, textile, or other substrate. For example, in particular embodiments, one or more actives is combined with a polymer (e.g., elastomer) to form a mixture or solution, which is applied to a yarn, yarn precursor, thread, filament, fiber, textile, or other substrate. In some embodiments, the final cross-linking (or all of the cross-linking, in some cases including polymerization) occurs in the presence of the dispersed or suspended active particles resulting in a configuration in which local stresses and strains on the polymer associated with “forcing” solid active particles into an already-cross-linked polymer (e.g., elastomer) are minimized or eliminated. Such strains, at least at high active loadings, can lead to higher permeability and loss of active-protecting effect. Entry of solid active particles (e.g., crystals) into, or formation inside, a previously cross-linked polymeric (e.g., elastomeric) core are also operable to cause distortion of the structure, leaving the active accessible when the purpose of encapsulation is to make it inaccessible. In other embodiments, however, all or a portion of the cross-linking occur prior to introduction of the active.

The delivery systems disclosed herein are operable to be used in a variety of applications. The applications discussed below are representative and illustrative, though certainly not all-inclusive. Suitable actives for use in the various applications are also provided below.

In one embodiment, at least one substrate (e.g., yarn, yarn precursor, thread, filament, fiber) is used to form a fabric or a textile. In certain embodiments the fabric or the textile formed with the yarn, yarn precursor, thread, filament, or fiber includes both the medicated yarn of this disclosure along with an ordinary, non-medicated yarn, yarn precursor, thread, filament, or fiber. For example, in woven textiles, the warp is operable to be traditional yarn and the weft is operable to be yarn of the present embodiments. In other embodiments, only medicated yarn, yarn precursor, thread, filament, or fiber is used.

In one embodiment, the at least one substrate includes at least one first substrate and at least one second substrate. The at least one first substrate includes at least one active composition and the at least one second substrate does not include at least one active composition. For example, a first yarn or filament includes a capsacinoid and a second yarn or filament does not include an active composition (i.e., is non-medicated). A fabric or textile is formed from the first yarn and the second yarn (e.g., knitted, woven, etc.).

In another embodiment, the at least one first substrate includes at least one first active composition and the at least one second substrate includes at least one second active composition. Advantageously, this embodiment allows for at least two active compositions. Further, the at least one first active composition and the at least one second active composition are operable to have different desired release rates. This embodiment allows for modifying the desired release rates of the at least one first active composition and the at least one second active composition by varying factors including, but not limited to, an occlusion percentage; a loading dose; a concentration of the at least one active composition; a particle size of the at least one active composition (e.g., a range of partible sizes); tension; composition of the yarn, yarn precursor, thread, filament, fiber, textile, or other substrate; exposed internal interstitial spaces of the yarn, yarn precursor, thread, filament, fiber, textile, or other substrate; the polymer, oligomer, and/or monomer matrix; characteristics of the polymer, oligomer, and/or monomer matrix. (e.g., density, porosity, hydrophobicity, hydrophilicity, thickness, degree of contact, nature of the polymer, oligomer, and/or monomer matrix (e.g., internal vs. external)); the at least one coating; and/or characteristics of the at least one coating (e.g., density, porosity, hydrophobicity, hydrophilicity, thickness, degree of contact, nature of the coating (e.g., internal vs. external)). For example, but not limitation, a textile is formed with a first yarn and a second yard. The first yarn includes a first active composition with a first occlusion percentage; a first loading dose; a first particle size of the at least one first active composition (e.g., a range of particle sizes); a first polymer, oligomer, and/or monomer matrix; and at least first one coating. The second yarn includes a second active composition with a second occlusion percentage; a second loading dose; a second particle size of the at least one second active composition (e.g., a range of particle sizes); a second polymer, oligomer, and/or monomer matrix; and at least one second coating.

Release rate is also impacted by temperature and exposure to fluids (e.g., sweat, sebum, etc.). Generally, a temperature of the article is between 15° C. and 30° C. Skin temperature is lower than core body temperature, and is usually between 33° C. and 37° C. internal body heat triggers sweating, which indirectly increases the release rate. Enclosure, such as in a shoe, is another way to create a favorable release environment.

While the examples described above include a first substrate and a second substrate, the present invention is not limited to two substrates. In one embodiment, the fabric or the textile includes a plurality of substrates. Each of the plurality of substrates is operable to include at least one active composition. In one embodiment, one or more of the plurality of substrates does not include at least one active composition. For example, but not limitation, a textile is formed with a first yarn, a second yarn, a third yarn, and a fourth yarn. The first yarn includes a first active composition; the second yarn includes a second active composition; the third yarn includes the first active composition, the second active composition, and a third active composition; and the fourth yarn does not contain an active composition. As one of ordinary skill in the art will understand, the factors listed above are operable to be manipulated to achieve a desired release profile.

In one embodiment, the textile formed with the yarn or filament is a knitted textile. In one embodiment, the knitted textile is a circular knit. Alternatively, the knitted textile is a flat knit. In one embodiment, the knitting of the textile is facilitated by treating the yarns of the present disclosure with a lubricant (e.g., 2% to 3% lubricant) prior to knitting.

Advantageously, the knitted textile is operable to provide both a relaxed state and a stretched state. In one embodiment, the wearable article or garment is formed of the knitted textile. In a preferred embodiment, the wearable article or garment is operable to substantially conform to at least one body part of a wearer. The at least one body part includes, but is not limited to, a finger, a hand, a wrist, an elbow, an arm, a shoulder, a torso, a head, a face, a neck, a toe, a foot, an ankle, a knee, a hip, and/or a leg. In one embodiment, the wearable article or garment produces a compressive force when worn. In one embodiment, the wearable article or garment is operable to provide compression to the at least one body part of the wearer. In one embodiment, the compression is between about 5 mmHg and about 60 mmHg. In another embodiment, the compression is between about 15 mmHg and about 50 mmHg. In yet another embodiment, the compression is between about 20 mmHg and about 40 mmHg. In one embodiment, the compression is between about 5 mmHg and about 15 mmHg, about 15 mmHg and about 20 mmHg, about 20 mmHg and about 30 mmHg, about 30 mmHg and about 40 mmHg, or about 40 mmHg and about 50 mmHg. In one embodiment, the wearable article or garment is seamless. Advantageously, a seamless wearable article or garment provides additional comfort to a wearer by reducing points of friction.

In some embodiments, the substrate is a yarn, yarn precursor, thread, filament, fiber, textile, or fabric. In certain embodiments, the yarn includes a nylon, polyester or acrylic material. In one embodiment, the wearable article forms an orthopedic cast, splint material, a wound dressing, socks, hats, face/ski masks, scarves, tiaras, chokers, skullcaps, undergarments, skin guards, wrist bands, arm bands, knee pads, bras, shirts, leggings, tights, nylon stockings, athletic supporters, protective athletic equipment (e.g., gloves, pads, and helmets), robes, neck bands, head bands, ear muffs, gloves, diapers, poultices, facial masks, paraffin gloves, and/or joint braces. In one embodiment, the present invention provides a wearable article including, but not limited to, an ankle sleeve, an arm sleeve, a calf sleeve, a knee sleeve, a lower leg sleeve, a wrist sleeve, a shirt or a partial shirt, pants or partial pants, leggings or partial leggings (e.g., ⅞ leggings, capri, shorts), an insole, a sock, or a glove. In one embodiment, the wearable article is a brace including, but not limited to, an ankle brace, an arm brace, a knee brace, a lower leg brace, a wrist brace, a finger brace, a shoulder brace, a neck brace, a back brace, or a hip brace. In one embodiment, the wearable article is a splint including, but not limited to, an ankle splint, an arm splint, a knee splint, a lower leg splint, a wrist splint, a finger splint, a shoulder splint, a neck splint, a back splint, or a hip splint.

In one embodiment, the wearable article is unitarily formed from the textile, the fabric, or the substrate. In one embodiment, the wearable article is operable to be pulled onto the at least one body part. For example, but not limitation, a knee sleeve is operable to be pulled over the foot and the calf, and placed over the knee. In another example, a shirt is operable to be pulled over the head, and arms of the wearer pulled through sleeves of the shirt.

In still another embodiment, the wearable article is operable to be wrapped around the at least one body part. For example, but not limitation, the wearable article is a back brace operable to secure around a torso of a wearer. In another example, the wearable article is an elastic bandage (e.g., ACE bandage) operable to wrap around the at least one body part (e.g., arm, leg).

In another embodiment, the textile, the fabric, or the substrate forms a layer in the wearable article. For example, and not limitation, in one embodiment, the textile, the fabric, or the substrate forms a first layer of the wearable article in contact with surface of the skin (e.g., a lining) and a second layer of the wearable article is exposed to the environment.

In certain embodiments, the yarn or substrate requires greater elasticity or stretch. Thus, in one embodiment, the yarn is plied or twisted with an air-covered yarn (e.g., spandex) to enable additional stretch of the yarn. Additionally, the yarn is operable to be air-covered/air-intermingled (i.e., blowing air onto the yarn and adding a spandex core into the middle of the yarn). These methods are particularly useful for garments that need a lot of stretch such as tights, leggings, or an elastic portion on a top of a sock or an ankle sleeve. In one embodiment, the wearable article is operable to stretch to conform to the at least one body part of a wearer.

In one embodiment, the wearable article further includes at least one closure mechanism. The at least one closure mechanism includes, but is not limited to, at least one strap, at least one snap, hook and loop tape, at least one tie, at least one buckle, at least one zipper, at least one lace, at least one closure system (e.g., BOA fit system), at least one latch, at least one hook, at least one elastic, at least one adhesive, at least one fastener, and/or at least one clip. In one embodiment, the at least one closure mechanism allows for customization of fit of the wearable article to the wearer. In another embodiment, the at least one closure mechanism secures the wearable article to at least one body part of the wearer. For example, but not limitation, hook tape and loop tape secure a wrist brace to a wrist of a wearer. In another example, the at least one closure mechanism is a tie to secure pants to a waist of a wearer. In still another example, at least one hook or at least one clip secures an elastic bandage (e.g., ACE bandage) to the at least one body part (e.g., arm, leg).

In one embodiment, the present invention provides an article (e.g., non-wearable article) that is operable to release at least one active composition. In one embodiment, the article is formed using yarns, yarn precursors, threads, filaments, fibers, textiles, and/or substrates (e.g., films, sheets, cut and sew pieces) that are operable to persistently release the at least one active composition. Advantageously, the article and/or the yarns, yarn precursors, threads, filaments, fibers, textiles, and/or substrates are operable to persistently release the at least one active composition after at least one wash cycle. In one embodiment, the yarns, yarn precursors, threads, filaments, fibers, textiles, and/or substrates form a knitted structure. Advantageously, the knitted structure is operable to provide both a relaxed state and a stretched state. In one embodiment, the article is formed of the knitted structure. Alternatively, the yarns, yarn precursors, threads, filaments, fibers, textiles, and/or substrates form a woven structure. In one embodiment, the yarns, yarn precursors, threads, filaments, fibers, textiles, and/or substrates are operable to be used for cut and sew. The article is operable to contact at least one body part of a wearer. In one embodiment, the article is operable to substantially conform to the at least one body part of a user. The at least one body part includes, but is not limited to, a finger, a hand, a wrist, an elbow, an arm, a shoulder, a torso, a head, a face, a neck, a toe, a foot, an ankle, a knee, a hip, and/or a leg. In one embodiment, the article is seamless. Advantageously, a seamless article provides additional comfort to a user by reducing points of friction. In an alternative embodiment, the article or garment is formed of a woven textile.

In one embodiment, the article includes a towel, a cover, a pillowcase, a sheet (e.g., flat sheet, fitted sheet), a comforter, a blanket, upholstery, a mat (e.g., yoga mat, exercise mat, anti-fatigue mat), a grip (e.g., athletic equipment, handlebars), and/or a sleeping bag or sleeping bag liner. In one embodiment, the cover includes a seat cover, an electric device cover (e.g., mouse cover, keyboard cover, phone cover), a heating pad cover, a duvet cover, ice pack cover, steering wheel cover, a furniture cover, and/or an exercise apparatus cover (e.g., bench cover). In one embodiment, the article is an upholstered article.

In one embodiment, the article further includes at least one closure mechanism. The at least one closure mechanism includes, but is not limited to, at least one strap, at least one snap, hook and loop tape, at least one tie, at least one buckle, at least one zipper, at least one lace, at least one closure system (e.g., BOA fit system), at least one latch, at least one hook, at least one elastic, at least one adhesive, at least one fastener, and/or at least one clip. In one embodiment, the at least one closure mechanism allows for customization of fit of the article to a platform (e.g., seat, steering wheel) and/or another article (e.g., pillow, duvet, mattress). In one embodiment, the article is operable to create contact with skin of a user via user to surface pressure. For example, but not limitation, at least one elastic is used to secure a fitted sheet to a mattress. In another example, at least one strap is used to secure a seat cover to a seat. In still another example, user to surface pressure is used to contact the article (e.g., seat pad) to the skin of the user.

A structural integrity of the wearable article and/or the article is preferably maintained after at least one wash. In a preferred embodiment, a size and/or a shape of the wearable article and/or the article does not substantially change after at least one wash. The wearable article and/or the article is preferably reusable as the wearable article and/or the article, respectively, after at least one wash. Prior art articles including superabsorbent polymers are not washable, and do not maintain their structural integrity after washing. For example, a disposable diaper including superabsorbent polymers cannot be reused as a diaper following at least one wash. The size and/or the shape of the diaper is substantially changed after at least one wash.

According to the present disclosure, large dosages of several grams or more per dosing, that are difficult to deliver as pills or in other dosing forms, are operable to be administered through skin-contacting material (e.g., clothing) in a way that is convenient, private, and even fashionable. Also, forgetful patients, such as schizophrenics, children, the elderly, Alzheimer's or pre-Alzheimer's sufferers, and the like can be assured of taking their medication (i.e., increased compliance) by virtue of simply lying on a pillow at night, or putting on their socks or another article of clothing such that they are in contact with the medicated material.

Importantly, long-term use of a transdermal approach is operable to be used without engendering the risks or downsides of occlusive and/or adherent patches or bandages. Certain areas of the body may be well-suited to delivery of an active substance via clothing, but not well-suited to more traditional methods of delivery. For example, the feet or hands are particularly well-suited to delivery via socks or gloves, whereas other topical delivery methods known in the art may not provide as efficient delivery because of the risk of being rubbed off, etc. Further, current fabric-based products of purported medicinal value, such as diabetic socks for example, which have not been provided with the obvious medicaments in the prior art due to washing requirements, are now operable with the present invention to be medicated and yet still remain fully washable.

Specific classes of compounds that are operable to be incorporated as actives and delivered include demulcents, emollients, lubricants, vasoconstrictors, antibiotics and antiseptics, antihistamines, immunosuppressants, local anesthetics, antiallergics, antifungals, vasoprotectants, anticoagulants, mucolytic and proteolytic compounds, antiglaucoma drugs, and anti-inflammatories, anesthetics, anti-helminthic, analgesics, steroids, non-steroidal inhibitors of the inflammatory cascade, anti-neoplastic, anti-angiogenic, calcineurin inhibitors, anti-ocular hypertensives, antivirals, antibacterials, neuroprotectants, anti-apoptotics, medications for dry eye, pupil dilating medications (mydriatics and cycloplegics), ocular decongestants, antioxidants, photosensitizers, photodynamic therapy agents, mast cell stabilizers, monoclonal antibodies, quinolone antibiotics, and intra-ocular pressure lowering agents. Specific ophthalmic pharmaceutical actives in addition to the above which are operable to be incorporated in the embodiments of the present disclosure are: acetazolamide, amikaci, anecortave, antazoline, apraclonidine, atropine sulfate, azelastine, azithromycin, bacitracin, bacitracin zinc, betaxolol hydrochloride, bimatoprost, brimonidine, brinzolamide, bupivicaine, carpbachol, carteolol hydrochloride, ceftazidime, ciprofloxacin hydrochloride, clindamycin, cromlyn, cyclopentolate hydrochloride, denufosol, dexamethasone, dexamethasone sodium phosphate, diclofenec sodium, dipivefrin hydrochloride, diquafosol, dorzolamide, doxycycine, edetate sodium, emadastine, epinastine hydrochloride, epinephrine, erythromycin, fluocinolone, 5 fuoruracil, fluoromethalone, fluoromethalone acetate, flurbiprofen sodium, fomivirsen, ganciclovir, gatifloxacin, gentimicin, gramicidin, imopenemn, ketotifen, ketrolac tromethamine, latanoprost, lerdelimumab, levocabastine, levofloxacin, levubunolol hydrochloride, lidocaine, lodoxamide, lotoprednol etabonate, medrysone, methazolamide, metipranolol, mitomycin, moxifloxacin, naphazoline, nedocromil, neomycin, ofloxacin, olopatadine, oxacillin, oxymetazoline hydrochloride, pegaptanib, pemirolast, pheniramine, phenylephrine hydrochloride, photofrin PIR 335, pilocarpine hydrochloride, polymixin B, prednisolone acetate, prednisolone sodium phosphate, proparacaine, ranibizumab, rimexolone, scopolamine hydrobromide, sulfacetamide sodium, tetracaine, tetrahydrozoline hydrochloride, timolol, timolol maeate, tobramycin sulfate, travoprost, triamcinolone acetonide, trimethoprim, tropicamide, unoprostone, urea, vancomycin, and verteporfin. Also suitable are derivatives, analogs, and prodrugs, and mixtures and combinations thereof.

In certain embodiments, actives that are dyed or colored are operable to be utilized. Colored actives provide several potential advantages, such as providing the user visual confirmation of activity, favorably modifying skin color or tone, and aiding in manufacturing QA/QC. Colored actives that are operable to be incorporated into yarns of the embodiments include, but are not limited to, Curcumin, Methylene Blue, Gentian Violet, Dantrolene sodium, and Oil Red O. These actives cover a range of therapeutic effects including anticancer, antibacterial, antifungal, antispasmotic, antioxidant, and/or anti-inflammatory effects.

An overview of various classes of conditions and treatments that are operable to utilize the various embodiments of the present disclosure are described below.

For application of actives to portions of skin suffering from abnormalities or for cosmetic improvement, the present embodiments offer direct skin contact, localizable coverage, washing machine compatibility (“washability”), rapid rate of release, continuous coverage through the night if desired or, as a patch, throughout the day or night. An example of an active for particular skin conditions includes tea tree oil for acne, eczema, psoriasis, etc. In addition to acne, other skin conditions for which the embodiments described here are particularly useful include rashes, skin allergies, folliculitis, impetigo, erysipelas, cellulitis, and dermatitis.

In applications that are considered therapeutic, cosmeceutic, cosmetic, etc., embodiments of the present disclosure are operable to improve skin condition and appearance via the release of, for example, vasodilators, rubefacients, ceramide, emollients, dermoprotective, lipolytic, or epithelializing compounds.

The embodiments described herein are operable to be of particular utility in medication- or antimicrobial-releasing socks, because socks must be washed so frequently, and the need is inherently high due to the relatively high rate of foot- and sock-related disorders, risks, and inconveniences, such as offending odors and the associated risks of infections (not only bacterial but also fungal and viral), and more serious risks faced by the growing incidence of diabetes.

In addition to acne, eczema and psoriasis, the following conditions are treatable, or preventable, with embodiments of the present disclosure: scleroderma (which often leads to Raynaud's syndrome), neutrophilic dermatosis, urticaria, xeroderma-pigmentosum, Goltz syndrome, recessive dystrophic epidermolysis bullosa, Harlequin ichthyosis, hypertrichosis, Morgellons disease, dermatofibrosarcoma protuberans, and infections such as human papilloma virus (HPV). Scleroderma may occur in both non-systemic and systemic forms, and while the delivery systems of the present disclosure are operable to be suited for treating the non-systemic form (e.g., with a fabric that would release an active oil extract from Salvia miltiorrhiza (Danshen) and/or from Capparis spinosa), they are effective against the systemic form as well. Salvia miltiorrhiza and Capparis spinosa work against scleroderma in two distinct mechanisms, so that delivery of a combination of the two oils via the delivery systems of the present disclosure are operable to be particularly efficacious.

In addition, delivery systems of the present disclosure are operable to provide for wound dressings that are non-adherent, non-occlusive for oxygen transport, and non-irritating. Wounds for which the systems are operable to be used include chronic wounds, such as malignancies, persistent infections (e.g., gangrene), decubitis, diabetic ulcers, and other ulcers of traumatic, venous, or ischemic origin. While the delivery system is operable to be used as a primary dressing, it is also operable to be effective as a secondary dressing, delivering medicament through the primary dressing.

In one embodiment related to wound dressing, a delivery system of the present disclosure is operable to be used as an insert or lining to a cast, splint, sling, or brace. There are over 6.8 million broken bones just in the U.S. every year, many requiring the use of a cast, splint, sling or brace for treatment. In the case of individuals treated for scoliosis, for example, patients must wear a full body cast and lie in bed for 3 to 6 months. There are many common negative issues associated with wearing casts for prolonged periods of time, including but not limited to, allergic reactions, skin sores, infections, joint stiffness, muscle loss, offensive odor, burns, and compartment syndrome, which greatly limits blood flow. Many or all of these negative side effects are operable to be effectively treated or mitigated by delivery of appropriate actives via the systems of the present disclosure. In one embodiment, such an application employs the disclosed systems in the form of an insert or lining to a cast, splint, sling, or brace. In one embodiment, the cast/insert system is designed such that the insert is operable to be removed (e.g., daily, if necessary) for washing without interfering with the supportive and protective functions of the cast or brace. The insert is operable to provide release of antimicrobials, growth factors, analgesics, and/or skin toning/cosmeceutical actives, and operable to release medicaments or essential oils designed to increase blood circulation. Several classes of actives are beneficial for treatment of wounds and are operable to be used with the systems of the present disclosure including, but not limited to, growth factors, clotting factors, local anesthetics, steroids, vitamins, minerals, antimicrobials, or (e.g., in milder wounds) antiseptics and bacteriostats.

Delivery systems of the present disclosure are operable to deliver sleep-/relaxation-aiding actives both into the bloodstream through release into the skin, and into the brain through the trigeminal neural pathway via nasal inhalation. Many compounds and oils from nature that induce relaxation often have analgesic action as well. Thus, due to action by these substances at one or more opioid receptors, embodiments of the disclosure are operable to be applied to release these actives and—potentially with combined transdermal and trigemical (inhalation) delivery routes—achieve a synergistic combination of anxiolytic and analgesic actions. In one embodiment, the present invention includes a combination of two actives. In one embodiment, the combination of two actives is a combination of lavender and Melissa essential oils. Plant essential oils that are purported analgesics include lavender, wintergreen, Roman chamomile, marjoram, peppermint, rosemary, thyme, vetiver, helichrysum, ginger, lemongrass, copaiba (copal), and balsam fir. Specific fractions or components of these oils, such as menthol, are operable to be used as well, particularly if they have substantial volatility. In some embodiments, the vapor pressure of the active at 35° C., for inhalation/trigeminal neural pathway delivery, is equal to or greater than about 0.01 Torr, greater than about 0.1 Torr, or greater than about 0.5 Torr. A drug with lower vapor pressure than this may still be practical if the potency of the drug is very high, such as with carfentanil.

Extracts and purified compounds from the following plants have been reported in the literature to have central-acting analgesic activity, and these are operable to be incorporated into the various embodiments of the present disclosure for relief of pain and, in many cases, for relaxation as well: Abutilon indicum, Acacia ferruginea, Acacia nilotica, Achillea ageratum, Acicarpha tribuloides, Aconitum carmichaelii, Aconitum flavum, Aconitum japonicum, Acorus calamus, Adansonia digitata, Afrormosia laxiflora, Agastache sinense, Ageratum conyzoides, Albizia lebbek, Alhagi maurorum, Aloe vera, Amelanchier ovalis, Anacardium occidentale, Anchomanes difforms, Annona squamosal, Apium graveolens, Araujia sericifera, Astragalus siculus, Baphia nitida, Berlinia grandiflora, Brassica rapa, Buddleja cordata, Bupleurum chinense, Cadia rubra, Caesalpinia ferrea, Calotropis procera, Cannabis sativa, Canthium parviflorum, Caralluma tuberculata, Carthamus tinctorius, Cedrus deodara, Celastrus paniculatus, Centella asiatica, Chasmanthera dependens, Chelidonium majus, Chrozophora verbascifolia, Cinnamomum zeylanicum, Citrullus colocynthis, Clematis chinensis, Cleome viscose, Clerodendrum infortunatum, Clitoria ternatea, Cocculus pendulus, Commiphora molmol, Cordia francisci, Cordia martinicensis, Cordia myxa, Cordia ulmifolia, Cucumis trigonus, Culcitium canascens, Curcuma zedoaria, Cuscuta chinensis, Cyathea nilgirensis, Cymbopogon schoenanthus, Cystoseira usneoides, Datisca cannabina, Desmodium canadense, Dioclea grandiflora, Diodia scandens, Dolichos falcatus, Ducrosia ismaelis, Egletes viscosa, Elaeagnus kologa, Elaeocarpus canitrus, Eriobotrya bengalensis, Ervatamia coronaria, Eryngium foetidum, Eucaluptus camaldulensis, Euphorbia hirta, Fagraea racemosa, Ficus glomerata, Foeniculum vulgare, Ganoderma lucidum, Genista patens, Glaucium flavum, Harpagophytum procumbens, Hedera rhombea, Heracleum hemsleyanum, Hibiscus sabdariffa, Himanthalia helongata, Himulus lupulus, Hypericum calycinum, Hypericum perforatum, Inula crithmoides, Inula viscosa, Ipomoea leari, Irvingia gabonensis, Juniperus oxycedrus, Laminaria achroleuca, Lantana camara, Lawsonia inermis, Ledebouriella seseloides, Lepidium sativum, Leucas aspera, Leucojum aestivum, Ligusticum sinense, Lippia alba, Lippia geminate, Luvunga scandens, Lycopodium clavatum, Lysimachia christinae, Maesa ramentacea, Melaleuca elliptica, Melaleuca styphelioides, Mentha piperita, Mikania cordata, Morinda citrifolia, Morus alba, Mucuna pruriens, Myrica nagi, Myrtus communis, Nepeta caesarea, Nepeta italica, Neurolaena lobata, Nigella sativa, Nyctanthes arbor-tristis, Ocimum sanctum, Oplopanax elatus, Origanum onites, Paeonia moutan, Panax ginseng, Pancratium maritimum, Paullinia cupana, Peganum harmala, Persea Americana, Photinia serrulata, Phyla nodiflora, Phyllanthus niruri, Phyllanthus sellowianus, Phyllanthus tenellus, Phyllanthus urinaria, Pimpinella anisum, Pinus koraiensis, Piper abutiloides, Piper cincinnatoris, Piper lindbergii, Piper longum, Piper methysticum, Piper umbellatum, Piscidia erythrina, Platycodon grandiflorum, Polygala cyparissias, Polypodium vulgare, Pongamia pinnata, Portulaca grandiflora, Portulaca oleracea, Prunus spinosa, Psammosilene tunicoides, Psidium pohlianum, Psychotria brachypodia, Psychotria colorata, Pterocarpus indicus, Ptychopetalum olacoides, Pycnocomon rutaefolia, Quercus infectoria, Quercus lineata, Randia siamensis, Ranunculus japonicas, Rhamnus procumbens, Rhazya stricta, Ricinus communis, Roylea elegans, Salvia haematodes, Santolina chamaecyparissus, Saussurea involucrate, Scabiosa atropurpurea, Senna italic, Serjania communis, Sida cordifolia, Sideritis mugronensis, Siphocampylus verticillatus, Stephania dinklagei, Stefania wightli, Strychnos nux-vomica, Synedrella nodiflora, Tabebuia chrysotricha, Tabernaemontana pandacaqui, Tamarix milotica, Taraxacum officinale, Teclea nobilis, Tecomella undulate, Teucrium carthaginense, Theobroma leiocarpa, Thymus vulgaris, Tillandsia usneoides, Tinospora cordifolia, Tinospora crispa, Torresea cearensis, Trachelospermum jasminoides, Trema guineensis, Trianthema portulacastrum, Tribulus terrestris, Trichilia catigua, Trigonella anguina, Trigonella foenum-graecum, Typhonium giganteum, Urtica dioica, Valeriana jatamansi, Vernonia condensate, Viola mandshurica, Vitex negundo, Zingiber officinale, and Ziziphus jujube.

The dissolution-limited embodiments of the present disclosure are operable to involve the use of a solid active ingredient as the active compound. One skilled in the art will recognize that, in many cases, the individual purified components of essential oils are often solids near ambient (room) temperature. For example, the liquid known as peppermint essential oil has as its predominant component menthol, which is a solid at room temperature. Menthol typically constitutes 50% to 80% of peppermint oil. As a further example, in a case where peppermint oil includes 70% menthol, the menthol component is accompanied by 30% of “other ingredients.” One of ordinary skill will understand that these other components are generally quite similar in molecular structure to menthol, but different enough that these minor ingredients act to lower the melting point of the menthol. One of ordinary skill in the art will further understand that this melting point depression effect can be common in plant oils, and means that many of the benefits from essential oils discussed in this disclosure in fact are operable to be achieved by solid actives, which are suited for the yarns and other substrates disclosed herein.

Fungal infections of the skin can be notoriously long-lasting, and compliance with an antifungal spray can be poor, for example, due to the need for daily application in the harried early morning time. An antifungal-medicated piece of clothing that is washable is operable to provide for long-term application to the site of infection without requiring any compliance on the part of the user, beyond the normal washing of the fabric that is required in any case. With, for example, 4 or 5 pairs of medicated socks, one could advantageously maintain continuous application of the active to the site during all waking hours of the day, and even at night if desired, without any conscious effort other than donning the designated socks each morning.

Vapor-releasing salves can be notoriously short-acting, and are not well suited for constancy of release. On the other hand, prior known patches are unsightly and even disfiguring. Advantageously, the embodiments of the present disclosure are operable to overcome these drawbacks by providing a sufficiently sophisticated delivery system for constancy of release which is nevertheless in the format of a fully functional (e.g., washable) article of clothing, such as a scarf, cap, veil, woven necklace, choker, neck band, ear muffs, or other headwear. Other applications that are operable to benefit from vapor release include trigeminal neuropathy, also known as “the suicide disease” due to the excruciating pain it causes. This condition is operable to be treated, for example, by using a delivery system disclosed herein that releases pain-numbing vapors such as menthol at a more constant rate than salves without requiring repeated applications every few hours. Other conditions treatable with such an approach include nasal congestion, emphysema, sarcoidosis, pleural effusion, pulmonary edema, pulmonary hypertension, pneumonia, tuberculosis, various infectious diseases, respiratory irritation (e.g., from breathing polluted air), and non-productive coughing.

Nutritional and nutraceutical compounds are also operable to be delivered transdermally according to embodiments of the present disclosure. Such compounds are operable to be delivered, e.g., via a transdermal patch or via everyday-use and other fabrics. Moreover, the large surface areas for transdermal delivery made possible by the delivery systems disclosed herein allow for delivery of larger doses than would be possible for traditional transdermal patches.

Considerable instruction is provided herein for producing washable, medicated materials for delivery of drug to the skin, which with many drugs translates into systemic delivery (i.e., transdermal delivery to the bloodstream). Nicotine, fentanyl, methylphenidate, scopolamine, nitroglycerine, rivastigmine, clonidine, Vitamin B12, estrogen and testosterone are some examples of drugs that are currently delivered transdermally through medicated patches, which are, of course, not washable, and thus must be discarded when dirty. Drugs requiring daily (or near-daily) application are operable to benefit from the embodiments described herein; for example, with children's ADHD, exposure to dirt of all forms is of course to be expected for a (hyperactive) child, and a washable, reusable patch is an advantage. Furthermore, if the present disclosure is used in the form of an article of clothing, particularly one that is fairly tight-fitting such as a sock or cap, then it becomes possible to eliminate the need for adhesives, which are essentially required for traditional transdermal patches and present a range of practical issues (e.g., allergic reactions). The embodiments described herein are also operable to be used to deliver drugs systemically through mucosal membranes—a route known as transmucosal.

In some embodiments, resiniferatoxin, and related materials containing components of greater than 1 billion Scoville units, including extracts of Euphorbia species such as Euphorbia resinifera or Euphorbia poissonii, are used as active compounds. Such compounds are operable to be used in treating pain and/or other conditions.

It is within the scope of this disclosure for the “active” to be one that improves the quality of life through the steady release, even through many washes, of a pleasant and social aroma, including pheromones. The designs discussed elsewhere herein for promoting release into the air (discussed above in relation to inhalation-based delivery) are operable to be used for such an application. Many of the essential oils listed and discussed herein are well established as pleasing aromas or even as perfume components. Some embodiments discussed herein that yield a more nearly-constant release rate are operable to be used to create textiles, such as dresses and scarfs, which do not suffer from the relatively short action of a single application (spray) of perfume, and in fact do not require any action on the part of the customer or user.

Delivery of drugs and even some nutritional supplements to infants and toddlers can be a challenge due to swallowing/coordination limitations and taste intolerance. The delivery systems disclosed herein provide convenient products and methods for overcoming these delivery challenges, by incorporating medicament- or supplement-releasing embodiments of the disclosure into and onto commonly used (and frequently washed) items such as pacifiers, milk/formula bottles, stuffed animals, etc. Hydrophobic actives, in particular, will in general be released more rapidly into milk or formula than into water, and milk, particularly flavored milk, is operable to mask the taste of medicaments, providing for relatively high dilutions without increasing total fluid intake.

In another embodiment, gloves releasing circulation-improving compounds or oils (e.g., vasodilatory, rubifacient) and/or local anesthetic compounds for treatment or prevention of Raynaud's disease and related conditions are provided.

Certain embodiments also provide athletic garments and undergarments and other sportswear/active wear releasing one or more of the following: performance-enhancing actives; aspirin, local anesthetic, and/or capsaicin or a capsaicinoid for relief of pain or cold; creatine, glutamine, citrulline malate, beta-alanine, and/or branched-chain amino acids for muscle recovery or muscle stimulation; and handkerchiefs releasing cologne or perfume, antimicrobials, and/or vitamins.

In certain embodiments, solid active particles or powders (e.g., crystalline active particles) are used. Solid active particles (e.g., crystalline active particles) are operable to be used, in part, to better achieve dissolution-limited release kinetics. Exemplary forms of active compounds that are operable to be used include, but are not limited to, crystalline or polycrystalline solid particles, semi-crystalline solid particles, amorphous solid particles, plant extracts comprising crystalline or amorphous solid domains of one or more active compounds from the plant, and mixtures or combinations thereof. In further embodiments, the active compounds are operable to include components or fractions of plant essential oils, which may be crystalline at room temperature, and suitable for use. The term “plant essential oils” is as described in U.S. Patent Application Publication No. 2014/0271863, which is incorporated herein by reference in its entirety and which also provides a listing of some of the organic compounds that provide for the desirable or therapeutic effects of these oils.

In some embodiments, the active includes a heating or cooling active. For example, in one embodiment, the active is selected from at least one of menthol, a menthol derivative, WS compounds (Wilkinson Sword™) (e.g., WS-3, 5, 12, and 23), methyl salicylate, ethyl salicylate, trolamine salicylate, capsaicin or a capsaicinoid, a synthetic heating or cooling agent (e.g., nonivamide), vanillyl butyl ether, ginger, eugenol, kunzea, arnica, camphor, niacinamide, and/or diphenyl hydramine. In certain embodiments, the active includes a cooling component blend (e.g., isopulegol, menthyl lactate, menthoxypropanediol, and 2-isopropyl-N,2,3-trimethylbutyramide (WS-23)). Other suitable heating or cooling actives are also within the scope of this disclosure.

In various embodiments, the active includes an anti-fungal active. For example, in one embodiment, the active is selected from at least one of clotrimazole, miconazole, ketoconazole, terbinafine, fluconazole, and/or amphotericin. Other suitable anti-fungal actives are also within the scope of this disclosure.

In some embodiments, the active includes an antipruritic and/or skin calming active. For example, in one embodiment, the active is selected from at least one of an antihistamine (e.g., diphenhydramine or hydroxyzine), a corticosteroid (e.g., hydrocortisone), a counterirritant (e.g., mint oil, menthol, or camphor), and/or a local anesthetic (e.g., lidocaine, pramoxine, benzocaine, trolamine, calamine, coenzyme Q-10, or diphenyl hydramine). Other suitable antipruritic and/or skin calming actives are also within the scope of this disclosure.

In certain embodiments, the active includes an acne-treating active. For example, in one embodiment, the active is selected from at least one of an alpha-hydroxy acid (AHA) (e.g., glycolic acid or lactic acid), benzoyl peroxide, clay, salicylic acid, sulfur, tea-tree oil, azelaic acid, topical retinoid, and/or kojic acid. Other suitable acne-treating actives are also within the scope of this disclosure.

In various embodiments, the active includes an emollient. In one embodiment, the emollient is selected from at least one of shea butter, cocoa butter, a castor oil derivative, lanolin, squalene, coconut, jojoba, sesame, almond, another plant oil and/or butter, cetyl alcohol and derivatives, and/or a stearate. Other suitable emollients are also within the scope of this disclosure.

Other suitable actives include, but are not limited to, cannabidiol, BEAUPLEX® VH; ALL-Q® (coenzyme Q10) plus; coenzyme, SPECIKARE™ CQ10; ROVISOME™ Q10; SIGNALINE™ S, SERENITYL™ BIOFUNCTIONAL; ATPEPTIDE™ IS; PROLIPID™ 141; VITAL ET™; GENTI-FOL® SA; CELLULINO™; lidocaine; SHAPEPERFECTION™; FRESH'N™ CC menthol 50% (CYCLOSYSTEM COMPLEX®); CARNIPURE™ CRYSTALLINE; SYNIORAGE™ LS 9847; ULTRA FILLING SPHERES™; RONACARE® nicotinamide; VEXEL™ SP; cafeisilane C; ROVISOME™ caffeine; ISOCELL™ SLIM; DISILANOL C+™; SYNIORAGE™ LS 9847, N,N-Diethyl-meta-toluamide (DEET), picaridin, and/or melatonin. Other suitable actives are also within the scope of this disclosure (see, e.g., Table 1).

TABLE 1

| | | Skin | |
|---|---|---|---|
| Skin | Skin | Energizing/ | Skin |
| Sensations | Calming | Renewal | Firming |
| Heat | Acne | Hydration | Anti-wrinkles |
| Cool | Irritation | Cellular Energy | Cellulite |
| Odor, Itch | Rosacea/ | Protection | Anti-Glycation |
| | Psoriasis | | |
| Capsaicin | Emollients | Vitamins A, C | Carnosine |
| | | and E | |
| Nonivamide | Lidocaine | Glycolic Acid+ | Carnitine |
| Menthol | CoQ-10 | Resveratrol | Vitamin Bs, E & C |
| WS Derivatives | Cinnamates | Polyphenols | Peptides |
| Miconazole | Piroctone | Niacinamide | Salicylates |
| | Olamine | | |
| Diphenhydramine | Calamine | Glycyrrhizinic | Alpha hydroxyl acids |
| | | Acid+ | |
| | | Ergothioneine | |
| | | Ferulic Acid | |
| | | Caffeine | |

The present disclosure provides drug-eluting yarns, yarn precursors, threads, filaments, fibers, textiles, and substrates that allow for ready integration into or use with existing commercial textile practices and materials. Highly desirable drug-delivery features such as near-zero-order release kinetics, high loading of active (e.g., drug), stabilization of active, and compatibility with various types of actives are also achieved. The embodiments disclosed herein are operable to be used for improving the health of skin via local delivery of dermatological actives, but are also capable of transdermal delivery of skin-permeable actives and numerous other applications.

Various methods for producing particles or powders of active are operable to be used. For example, methods for producing small crystals of an active compound are operable to be categorized according to whether larger starting materials are milled down to smaller size (the “top-down” approach), or microscopic crystals are engineered from the start (the “bottom-up” approach). Methods for milling include high-shear homogenization, high-pressure homogenization (also known as microfluidization), ultrasonication, wet milling, ball milling, and others. “Bottom-up” methods generally rely on precipitation or crystallization in the presence of size-reductive methods such as homogenization and sonication. Active compounds are also operable to be crystallized within microstructures, such as emulsion droplets, liposomes, microparticles, etc., that are operable to limit the size of the resulting crystals.

The active particles (e.g., active crystals) are operable to be dispersed or immobilized in various types of cross-linked, hydrophobic polymer matrices. For example, in some embodiments, the polymer matrix includes an elastomer in which the active particles are dispersed. Exemplary elastomers include, but are not limited to, silicones, rubbers, halogenated rubbers, polyether block amides, ethylene vinyl acetates, elastolefins, polyurethane elastomers, fluoropolymer elastomers (fluoroelastomers), which are also operable to repel hydrocarbons, thermoplastic elastomers (TPEs), and mixtures and combinations thereof. The polymer matrix is also operable to include an elastomer blended or otherwise mixed with other polymers. In such embodiments, the elastomer domains are operable to be continuous from one end of the elastomer domain to another end such that active particles dispersed within the elastomeric domains are operable to move or diffuse from one end to the other. For example, an illustrative polymer matrix is operable to include both elastomeric domains and crystalline domains, where the elastomeric domains are in continuous communication with one another.

Some of the embodiments disclosed herein include cross-linking so as to lock, hold, or otherwise temporarily retain particles (e.g., crystals) of an active in place and protect the particles from degradation and premature loss, particularly in the face of stress conditions such as those encountered in laundering.

In certain embodiments, polymers (e.g., elastomers) that have been cross-linked in the presence of dispersed or suspended active are coated along the longitudinal axis or the length of the yarn or substrate. In specific embodiments, polymers (e.g., elastomers) that have been cross-linked in the presence of dispersed or suspended active are intermittently or partially coated along the longitudinal axis or the length of the yarn or substrate. As is more fully described below, mathematical equations given herein prescribe the architecture for yarns, yarn precursors, threads, filaments, fibers, textiles, and substrates that yield long-duration release with zero-order or near-zero-order release kinetics. For example, one of these mathematical conditions determined by these equations puts a limit on the length of the coated (or more precisely, “occluded”) segments of a yarn or substrate, which should not be too long; otherwise, the time needed for an active particle to diffuse to a non-occluded region will be too long to achieve the desired release profile.

The present disclosure provides drug-eluting yarns, yarn precursors, threads, filaments, fibers, textiles, and substrates that allow for ready integration into or use with existing commercial textile practices and materials. Highly desirable drug-delivery features such as zero order or near-zero-order release kinetics, high loading of active (e.g., drug), stabilization of active, and compatibility with various types of actives are also achieved. The embodiments disclosed herein are operable to be used for improving the health of skin via local delivery of dermatological actives, but are also capable of transdermal delivery of skin-permeable actives and numerous other applications, as described in greater detail below.

In some embodiments, the present invention uses yarns formed using extruded fibers. For example, synthetic yarns (e.g., nylon, polyester, etc.) are operable to include extruded fibers. As further detailed below, the active compound and/or the polymer matrix is also operable to be mixed and extruded with the yarn precursor (e.g., nylon or polyester polymers) during formation of the extruded fibers. Further, some embodiments provide active-loaded yarns and substrates wherein the active is in a substantially inert and/or protected state (e.g., crystalline form) and is also protected against degradation by the use of materials that are operable to be processed at room temperature. For example, room-temperature vulcanizing (RTV) polymers and elastomers are operable to be used as materials for the polymer matrix. In such embodiments, wasteful release of active is operable to be limited, at least in part, by 1) the immobilization of active (e.g., crystalline active) within a polymer matrix (e.g., an elastomer matrix) that exhibits negligible or no swelling with water; 2) the coating; and 3) the relatively small proportion of time spent in conditions of wasteful release, such as during laundering of the yarn or substrate.

In particular embodiments, the delivery system disclosed herein includes a yarn, yarn precursor, thread, filament, fiber, textile, or substrate that includes an active compound that is dispersed or suspended in a polymeric (e.g., elastomeric) matrix, and the yarn, yarn precursor, thread, filament, fiber, textile, or substrate containing the active compound and the polymeric matrix is partially or substantially coated or occluded by a coating material that is impermeable or substantially impermeable to the active compound, such that the delivery system provides a dissolution-limited release of the active upon application. In specific embodiments, the percentage of the coated or occluded area, segment, or region that restricts the release of active from the active-loaded polymer (e.g., elastomer) matrix is operable to be between about 80% and about 99.999%, between about 90% and about 99.995%, between about 95% and about 99.99%, between about 95% and about 99%. This particular embodiment of a yarn, yarn precursor, thread, filament, fiber, textile, or substrate that is substantially coated results in release of the active through a relatively small area, thus allowing for extended release of the active over an extended period of time. In some embodiments where a “burst” release is desirable or acceptable, or where rapid release of active is desired even at the expense of constancy of release rate, a coated/occluded percentage of less than 80% is operable to be invoked.

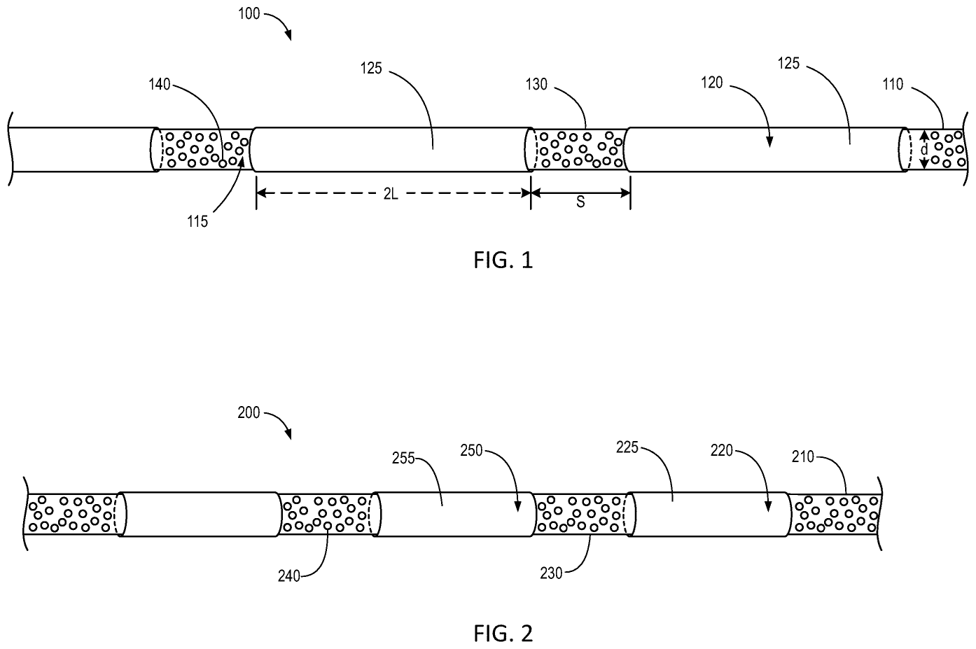

Shown in FIG. 1 is an embodiment of a drug delivery system 100 of the present disclosure. As can be appreciated, although much of the disclosure and Figures may refer to or depict a yarn, other substrates (e.g., yarn precursors, threads, fibers, etc.) are also operable to be used in an analogous manner. The drug delivery system 100 includes the yarn, yarn precursor, thread, filament, fiber, or substrate, which can also be referred to as the core 110 of the drug delivery system 100. A polymer (e.g., elastomer) is operable to be incorporated or loaded into the core 110 to form a polymeric (e.g., elastomeric) matrix 115, which may also be referred to as an inner matrix, inner polymer matrix, or drug matrix. The core 110 is also operable to include active compounds or particles 140 that are dispersed and/or immobilized in the polymer (e.g., elastomer) matrix 115 of the core 110. In certain embodiments, the polymer (e.g., elastomer) and/or the active 140 is applied to the core 110 of drug delivery system 100. Segments of the core 110 are operable to be coated, partially coated, or uncoated. In particular embodiments, the core 110 of the drug delivery system 100 is operable to be partially, selectively, or intermittently coated along the longitudinal axis or length of the core 110. For example, as illustrated in FIG. 1, the core 110 is operable to be intermittently coated with a coating 120 that is impermeable or substantially impermeable to the active 140 in the inner polymer matrix 115. Because the coated or occluded segments 125 of the core 110 are impermeable or substantially impermeable to the active 140 loaded into the drug delivery system 100, they are also referred to herein as “occluded” segments. In one embodiment, the core 110 similarly includes exposed, uncoated, non-occluded, or “open” segments 130, which are permeable to the active 140.

As is also shown in FIG. 1, the coated or occluded segments have a length of 2 L, while the uncoated or non-occluded segments have a length of S. The diameter of the core is represented by d. In one embodiment, the occluded segments 125 are operable to be configured such that the ratio of 2 L/d is larger than about 5, larger than about 10, or larger than about 25. Similarly, the ratio 2 L/S of adjacent occluded and non-occluded segments (125, 130, respectively) is operable to be greater than about 1, greater than about 4 (corresponding to 80% occlusion, 20% open), or greater than about 9 (corresponding to 90% occlusion, 10% open). Adjacent occluded and non-occluded segments are operable to refer to segments that are next to each other along the longitudinal axis of the yarn or core 110. In certain embodiments, the drug delivery system 100 are operable to be configured such that the lengths 2 L and S of occluded and non-occluded segments (125, 130, respectively) are substantially constant or uniform along the length or longitudinal axis of the yarn or core 110. In other embodiments, the lengths 2 L and S of occluded and non-occluded segments (125, 130, respectively) are operable to be varied along the length or longitudinal axis of the yarn or core 110.

Referring to FIG. 2, in certain embodiments of a drug delivery system 200, more than one type of occluded segment 225, 255 is operable to be provided. For example, the core 210 is operable to be coated with a first coating 220 and a second coating 250, each of which is operable to be impermeable, substantially impermeable, or semi-permeable to the active 240. Additional coatings with various functional and physical properties are also operable to be employed (e.g., a third coating, fourth coating, etc.). Coatings 220 and 250 are operable to be configured in any suitable arrangement. For example, they are operable to be adjacent to each other or they are operable to be separated by a non-occluded segment 230, or a combination thereof. In certain embodiments, coatings 220 and 250 are operable to be arranged such that moving axially along the length of the yarn or core 210, one would encounter segments alternating between two or more polymer coatings (e.g., polymer A and polymer B). Uncoated segments 230 are also operable to be included as part of the arrangement. As described more fully below, the pattern and sizing of the coated segments are operable to be selected to control the rate of release of the active 240 from the drug delivery system 200 over time.

In particular embodiments, the coatings 220 and 250 are operable to include different materials with different properties. For example, the coatings 220 and 250 are operable to contain polymers having different properties that will affect the rate of release of active 240. For example, in one embodiment, polymer B is more soluble in water or other aqueous milieu than polymer A, so that the release rate of the active 240 is relatively low until faster release is “triggered” or commenced by exposure to water (e.g., one or more launderings or rinses, or sweat) that breaks down or degrades the polymer B segments to expose the active-containing core 210. Such degradable materials are known in the art, such as water-soluble polymers, poly-lactic acid, poly-L-lactide, poly-glycolic acid and their copolymers, as well as other polyesters, polycaprolactone, biopolymers such as those based on collagen or gelatin or other peptides, certain natural gums, certain polysaccharides, chitosan and derivatives, and derivatives and mixtures thereof. Other erodible or biodegradable polymers are also operable to be used.

In other embodiments, two different polymers that are each impermeable to a different type of active compound 240 are operable to be used and arranged in a manner that controls the rate of release of each of the different active compounds 240. Furthermore, in additional embodiments, three or more coatings (e.g., polymers A, B and C) are also operable to be used and arranged in a variety of configurations (e.g., alternating) and with or without uncoated segments.

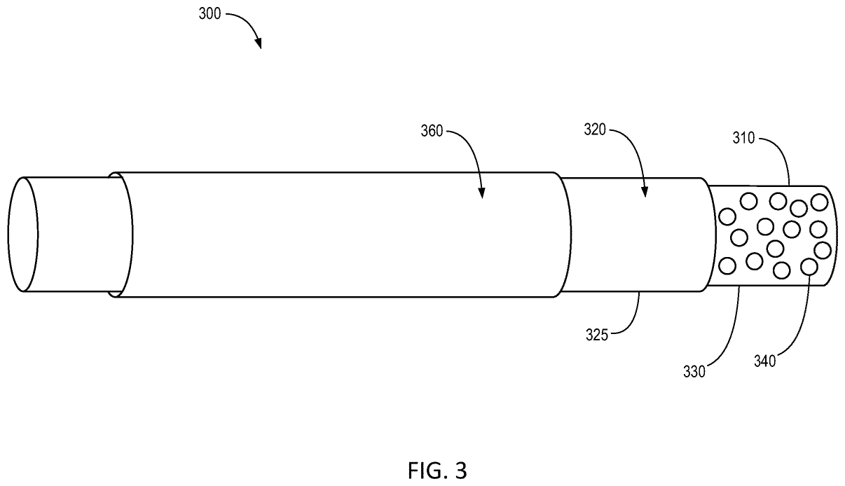

Referring to FIG. 3, a drug delivery system 300 is shown having an outer sheath 360 that covers both the occluded segments 325 (i.e., covered with a coating 320) and open segments 330 of the yarn or core 310. The outer sheath 360 is operable to cover the entire length of the yarn or substrate 310, or one or more selected portions thereof. In some embodiments, the outer sheath 360 includes a material that breaks down or degrades over time or upon exposure to a “trigger” or particular event (e.g., exposure of a water-soluble sheath to water or sweat), thereby leaving the underlying yarn or substrate including coated and/or uncoated segments (325, 330, respectively) as described above. In some of such embodiments, the outer sheath 360 is operable to be impermeable or substantially impermeable to the active 340 such that it prevents release of the active 340 until a “trigger” event, at which point the release rate is operable to be controlled by the arrangement of the coated and uncoated segments (325, 330, respectively) underlying the outer sheath 360. The presence of the outer sheath 360 is operable to provide additional adjustments to the desired release of the active 340 over time, including the potential for controlled or delayed release of the active 340 from the drug delivery system 300.

Various materials are operable to be used to prepare the polymeric or inner or protective matrix of the drug delivery system. For example, the matrix is operable to include a polymer or an elastomer that exhibits relatively low toxicity, low allergenic potential, and/or low skin irritation. The matrix also is operable to release the active at a rate that delivers an efficacious and reasonably safe dose in the time anticipated or desired for the drug delivery system-tissue contact. Additional details regarding the matrix are found in U.S. Patent Publication No. 20200390720, which is incorporated herein by reference in its entirety.

In some embodiments, the “coating” or “sheath” materials that occlude the active-in-matrix dispersion in the embodiments of this disclosure are of low permeability or impermeable to the active. Many commercial coatings well known to one skilled in the art are operable to be used, with consideration to surface interactions. The coating is operable to be inorganic or organic, or a combination of, for example, inorganic particles or laminates bound together with an organic polymer as binder. The coating is operable to be an inorganic coating, such as a composition of zinc oxide (e.g., 93% zinc oxide), as used in an example provided herein. The coating is also operable to be selected from an organic polymer.

Low permeability is often be associated with a highly crystalline polymer, though high crystallinity is not necessarily required if the polymer is in the glassy state near ambient temperatures. In some embodiments, polymers of low crystallinity that nonetheless have high tenacity and low permeability to one or more actives are operable to be used as coatings.

In other embodiments, the coating material includes a high-crystallinity thermoplastic polymer and is processed thermoplastically. In certain embodiments of the present disclosure, the melting temperature of the coating polymer is low enough to allow processing at temperatures that are low enough to limit thermal degradation of the active. Coating materials are operable to be purchased commercially, or are operable to be prepared by dissolving the desired polymer in a suitable solvent. Exemplary polymers for use as coating materials include polypropylene, polyvinyl chloride, PTFE (non-porous), polyvinylidene fluoride (PVDF), PMMA, shellac, polycarbonate (e.g., Lexan), polybutylene terephthalate, epoxy, polyethylene terephthalate (PET), high-density polyethylene, nylon, polyimide, celluloid, acrylonitrile butadiene styrene (ABS), phenol-formaldehyde resin, and polystyrene. Additional details regarding coatings compatible with the present invention are described in U.S. Patent Publication No. 20200390720, which is incorporated herein by reference in its entirety.

While it is possible for a lower surface energy coating to creep over a higher energy inner matrix so as to occlude the desired non-occluded surface (e.g., the end of a yarn, substrate, or core), this can be prevented. For example, one way to prevent such creeping is to select two polymers with the correct order of surface energies (many elastomers are of low surface energy, e.g., polysiloxanes). Another way is to take advantage of the high modulus of the polymers that one could choose for the sheath or coating polymer, which are operable to exhibit high crystallinity, and arrange the processing conditions such that any tendencies to migrate are limited by the time spent in the molten state.

Embodiments of this disclosure provide yarns, yarn precursors, threads, filaments, fibers, textiles, or substrates that release at a constant or near-constant rate over most of the duration of an extended-release profile, the constancy of release being due to the substantially dissolution-limited nature of the release mechanism (described more fully below), and the extended lifetime of release being enabled by the restriction of the non-occluded area over which release is operable to occur from the inner, active-loaded polymer. The percentage of occluded area restricting release of active from the active-loaded inner polymer is operable to be between about 80% and about 99.999%, between about 90% and about 99.995%, between about 95% and about 99.99%, or between about 95% and about 99%. Phrased in terms of non-occluded (“open” or “exposed”) regions, the percentage of non-occluded area through which release of active from the active-loaded inner polymer without interference from the coating is operable to be between about 0.001% and about 20%, between about 0.005% and about 10%, between about 0.01% and about 5% or between about 1% and about 5%. Generally, more demanding applications requiring exacting release kinetics call for a lower open fraction.

As discussed above, the embodiments of this disclosure are operable to be configured to achieve a constant or near-constant rate of release of an active compound from the delivery system. This is of particular value for an active that has a relatively low therapeutic index such that systemic levels should be kept as constant as possible over time, or when the diffusion-limited t^{1/2} profile would waste much of the active during the early-time high release rate.

Certain embodiments of the disclosure rely substantially, or even entirely, on the release characteristics of the polymer matrix that is in direct contact with the active. As described above, the embodiments described herein are operable to include solid active (e.g., crystalline active, active powders, etc.) dispersed in a polymeric matrix, and configured such that the egress of active from the matrix is substantially limited by the appropriate shape and coating of the polymeric matrix, so as to achieve a near-constant rate of active release over an extended period of time. Additional details regarding active release are described in U.S. Patent Publication No. 20200390720, which is incorporated herein by reference in its entirety.

Referring to FIG. 1, if D is the diffusion rate of the active in the yarn or substrate, K is the dissolution constant of the active in the polymer matrix, R is the effective radius of a constituent fiber of the yarn, A is half the surface area of the open section of length S so that A=πRS, and the volume of a fundamental repeat unit is πR²(L+S/2), which is approximately πR²L since S<<2 L. C₀ is the initial concentration of active in the polymer matrix (including dissolved and undissolved), and C_{S} is the saturation concentration of the active in the polymer matrix.

If the open segments of the yarn are (or remain) bulked after treatment with the polymer, oligomer, or monomer matrix (and coating) (as described below), then, mathematically, this is equivalent to a small value of R indicative of the radius of the substituent fibers, and a higher value of N indicating the (average) number of these same substituent fibers.

The variable N gives the number of constituent fibers of radius R in the cross-section of the yarn, and as would be clear to one skilled in the art, depending on the yarn structure this could be the number of filaments in a multifilament yarn, the number of plies in a twist, the (average) number of independent strands in a bulked yarn, and so forth. The values of N and of the fundamental unit radius R is operable to be defined consistently such that the approximation of the cross-section of the yarn as N circular discs of radius R is a reasonable one. In some instances, the cross-sectional structure in the uncoated, “open” regions can be quite different from that in the coated (occluded) regions. The subscript “1” corresponds to the open regions and “2” to the occluded regions in this disclosure.

With this nomenclature, and as to embodiments wherein S<<L, the following approximate equation for the release rate (flux) of active per unit length (here, per centimeter) of yarn holds to within a constant numeric and dimensionless factor:

Q=Cₛ(DK)^{1/2}R₁SN₁/L

This equation gives the release rate at steady-state when in the dissolution-limited case, exact conditions for which are given herein. The release rate equation is most easily interpreted when the entire “open” area on the yarn is abutting a receiving surface such as skin or mucosal tissue. Because only a portion of a given yarn will be touching or contacting skin, the equation represents a maximal release rate that is to be multiplied by the fractional open area that is touching or contacting skin or another receiving medium.

Since the volume-weighted average concentration is C₀ (which includes both dissolved and undissolved active), and again assuming S<<L, we have the approximate expression for M, the total mass of active released over the entire release profile:

M=C₀R₂²N₂

The duration of release T is then:

T=M/Q=(L/S)·(N₂/N₁)·(R₂²/R₁)·(C₀/C_{S})/(DK)^{1/2}

which, in the case where the fibrillar structure is approximately the same in the coated regions as in the open regions, simplifies to:

T=M/Q=(L/S)·R·(C₀/C_{S})/(DK)^{1/2}

In most cases, even if the open and occluded regions have very different fibrillar structures, the total cross-sectional area will nevertheless be the same in the open and occluded regions, even if the fibrils in the occluded regions are “glued” together by the coating so that N is reduced (often to 1). In these cases, the following equation can be used, derived by setting the total cross-sectional areas over all fibrils equal in coated and open regions:

N₁·R₁²=N₂·R₂²

And using this relationship, the following equation can be written:

T=(L/S)·R·(C₀/C_{S})/(DK)^{1/2}

This equation, which holds quite broadly (unless the open regions are engineered to be a different total cross-sectional area of the core matrix) tells us that the internal/fibrillar structure inside the coated regions—which is operable to be fibrillated and bulked even after coating—does not substantially affect the duration of release. This is a result of the fact that diffusion in the long (L>>R₂), coated segments is very closely approximated as a one-dimensional process whether there is bulk (N₂>1) or not (N₂=1).

From these equations, it can be seen that the duration of release T is operable to depend on more than one structural dimension. In particular, it is operable to depend on the term (L/S)·R₁.

As an example of kinetic control, the degree of bulking after treatment with the polymer, oligomer, or monomer matrix is operable to be adjusted by adjusting the tension on the yarn during treatment with the polymer, oligomer, or monomer matrix and curing. Open regions of the final yarn will maintain this bulk if handled properly, because they need not be exposed to the coating. This tension-adjusted bulking is operable to greatly reduce R₁ and therefore the term (L/S)·R₁. This could strongly affect the duration of release T. In general terms, the small thicknesses, in 2 dimensions, of yarn, and particularly of fibrils, means that surface-to-volume ratios will be higher than in volumetric or even thin-film configurations. For a given volume of active-loaded matrix, a higher surface area of open regions means that release rates (Q) are relatively higher and duration of release (T) lower. In order to achieve longer durations, the most efficient way is generally to reduce surface areas by decreasing the open length S. In some cases, in order to obtain desired release characteristics, it might be necessary to reduce S to only a few hundred microns.

Because the release rate does not depend on L, whereas the duration does, the duration of release is operable to be controlled by adjusting the length of the occluded segments without affecting the rate of release; this is because at steady-state, the active concentration is at the saturated value C_{S} regardless of L (recognizing that this steady state lasts longer as L increases). In short, the present disclosure provides not only for near-constant drug release, but also for independent control of release rate Q and duration of release T. This is an important advantage of the present disclosure because, in practice, the choice of the polymer that forms the inner matrix is driven by many factors other than D and K, such as cost, ductility, processing ability, cross-linking considerations, tack/adhesion, etc. Thus, one does not want to be restricted in polymer selection in order to meet kinetics requirements (D and K) without an easily adjustable parameter such as the aspect ratio of the yarn or substrate.

Diffusional distances, represented by the occluded segment length L in the present disclosure, are operable to be much longer than those represented by the film thickness in other types of structures. Thus, for the same chemistry (i.e., the same values of D and K), the duration of action is operable to be very long—an inherent advantage of the embodiments described herein—as compared to a traditional nonwoven patch. However, in one embodiment, increasing S is operable to counteract this effect.

It is emphasized that in this disclosure, C₀ is the volume-averaged concentration of active within the cross-linked polymer or elastomer including both the dissolved and undissolved active. The ratio C₀/C_{S} is operable to be at least about 5, or greater than or equal to about 10. The matrix is operable to be heavily “supersaturated” in view of the large amount of crystalline material, relative to the active that is dissolved in the matrix at the time of first use (the latter of which can lead to an exaggerated burst effect if the ratio C₀/C_{S} is not large enough).

In the practice of the embodiments of this disclosure, particularly in embodiments where the final yarn is bulked in the open segments, the very small value of R₁, measured literally in 10s of microns in many cases, will mean that S may need to be small in order to reach a targeted duration T; the desired length S might be as low as 100 microns. In one embodiment, the best processing method for adding the coating is to physically clasp or adhere ring-shaped masking solids; simply applying a screen that does not contact the yarn may not be sufficient to control the spread (or variability thereof) of coating to within the required precision.

As is stated above, embodiments of this disclosure are operable to provide yarns, yarn precursors, threads, filaments, fibers, textiles, or substrates that release at a near-constant rate per area of skin contact over a dominant portion of the duration of an extended release profile, the constancy of release arising from the dissolution-limited nature of the release mechanism, which in turn results from conformance to the following mathematical conditions, where D is the diffusion rate and K the dissolution constant of the active in the core, and u=1 centimeter is a standard unit of length:

1. The ratio D/(K·u) is greater than about 10, greater than about 30, or greater than about 100;

2. The ratio KLR₁/(SD)=(LR₁/S)·(K/D) is less than about 0.1, less than about 0.025, or less than about 0.01; and

3. The ratio 2 L/S is between about 4 and about 30,000, between about 9 and about 10,000, or between about 99 and about 3,000.

Dimensionality is operable to be manipulated in a very surprising way in the present disclosure, as can be demonstrated with reference to the three conditions noted above. The following compares 2-D and 1-D cases of when an intermittent or patterned coating is used to provide predominantly linear release through the “occlusion-dominance” approach discussed in the previous paragraphs. In a patch of material in which the matrix for the active is not woven, e.g., a gel, liquid, or nonwoven polymer film, then within the “mostly occluded” approach disclosed herein, the diffusion of the active will quite generally be primarily in two dimensions (“2-D”), namely in the plane of the thin film. The patterns of occluded/non-occluded regions are operable to fall into two classes:

2-D pattern: a pattern that is repeating in two dimensions and conforms to one plane group listed along with the 230 space groups in the International Tables of Crystallography.

1-D pattern: there is only a one-dimensional “line group.” The only such pattern in the present context consists of alternating occluded and non-occluded segments, each of fixed width.

In some embodiments, the 2-D pattern is inherently inferior. For example, the 2-D pattern case can be simplified to a fundamental “cell,” best viewed as, e.g., a hexagon, wherein a particular hexagon is made up of all points that are closer to a particular node than to any other node. In the present context, each node represents the center of a non-occluded (or “open”) region. By analyzing a single representative hexagon, the result is easily extended to the entire plane. Key to the issue of dimensionality is that the proportion, f, of non-occluded area varies as the square of the radius, measured outward from the nodal center of the open region:

f=(3/π)(Rₒ/R_{H})²

where Rₒ is the radius of the open region and R_{H} is the radius of the circle that circumscribes the hexagon. Functionally, R_{H} is a measure of the diffusional length, over which concentration gradients drive movement of active particles, e.g., from crystals in occluded regions to release at open regions. From the point of view of maintaining zero-order release kinetics predominantly over the release profile, the ratio Rₒ/R_{H} is operable to be less than about 0.2, or less than or equal to about 0.1; but, from an area-coverage perspective, the areal fraction (Rₒ/R_{H})² is operable to be much higher than the about 0.01 to about 0.04 that corresponds to Rₒ/R_{H} values of about 0.1 and about 0.2, respectively.

Thus, according to this approximate analysis, if one were to use a 2-D pattern in which each diffusional path to a node was, for example, 90% occluded—as is desirable for maintaining a high linearity in the release profile—then the “open”/non-occluded fraction would be under 1%. A value this low would be generally unacceptable for delivery to the skin; even if having only 1% of the area active is consistent with a desired (high-potency) drug, the end-user will experience “spotty” delivery over the desired area of coverage. In some cases, this may be acceptable, but in general the sensitive nature of skin would not allow for spotty coverage to this extent. One can anticipate that a significant fraction of people would develop some sort of discoloration if relatively large amounts of drug were focused on a few small dots of contact.

Instead, utilizing the simple “1-D pattern” approach, namely, alternating occluded and non-occluded stripes (or strips, rows, etc.), then the ratio 2 L/S provides both the real fraction of open regions and the open fraction of the diffusional/gradient length; one does not square the ratio as in the 2-D case. Thus, a “striped” pattern allows adjustment of the coating pattern to achieve a quasi-zero-order kinetic profile without having to sacrifice the fraction of open area for release. Furthermore, in one embodiment, the stripes are arranged perpendicular to the main longitudinal axis of the yarn or at any angle thereto, or a combination of both.

Additional details regarding making or manufacturing the delivery systems are disclosed in U.S. Patent Publication No. 20200390720, which is incorporated herein by reference in its entirety.

Coatings and sheaths of the embodiments of this disclosure are operable to be applied to substrates including at least one active composition via one of two general processes. Coating fluid is operable to be applied continuously or intermittently to a substrate (e.g., yarn, filament), or the substrates operable to be intermittently “masked,” with small masking pieces (e.g., clasps or tape, etc.) that are applied to the substrate prior to coating and removed afterward.

For example, an intermittent coating is operable to be achieved by attaching relatively small or tiny clamps to yarn, for example, at regular intervals before the yarn undergoes coating. Another method for blocking or “masking” the yarn from coating over specific stretches of yarn—ultimately the “open” regions of the final yarn—is to coat these regions with a polymer or powder that either substantially repels the subsequently applied coating, or substantially removes the coating when it is removed, e.g., the removal being affected by dissolution into water or solvent, air impingement, or in some cases, simple bending or twisting.

In some embodiments, the monomer resin system is operable to be applied to or disposed on a single filament yarn such that the single filament yarn is coated with the monomer resin system. In certain embodiments, the monomer resin system is operable to be applied to, disposed on, or incorporated into a multiple filament yarn (i.e., a yarn including a plurality of single filaments). The monomer resin system is operable to coat and/or be applied to the multiple filament yarn. Stated another way, the monomer resin system is operable to coat or cover at least a portion of an outside surface of the multiple filament yarn and/or the monomer resin system is operable to be absorbed or soaked up by the multiple filament yarn such that at least a portion of the monomer resin system is disposed in the interstices of or spaces between the plurality of single filaments that make up the multiple filament yarn. In various embodiments, a single filament yarn and/or a multiple filament yarn is operable to be treated with a monomer resin system to form a drug delivery system.

The resultant monomer resin system is operable to be transferred to a coating line or application line for delivery to a yarn, fabric, or substrate. In certain embodiments, the monomer resin system treated or resultant treated fiber or yarn is operable to then pass through a curing chamber or light box including a series of UV LED lights tuned to the wavelength of the initiator to activate the initiator. Other lighting sources are also within the scope of this disclosure.

One aspect of the present disclosure is related to methods for making or manufacturing a drug delivery system. The methods are operable to include forming a solution including a monomer, an initiator, and/or an active compound. The methods are also operable to include applying or disposing the solution on a substrate, wherein the substrate includes at least one of a yarn, yarn precursor, thread, filament, fiber, textile, and/or another suitable substrate. In some embodiments, the methods include exposing the solution and the substrate to UV light to initiate polymerization and/or cross-linking of the solution. In one embodiment, the solution further includes an oligomer and/or any other suitable material or component.

Another aspect of the present disclosure is related to drug delivery systems. In one embodiment, the drug delivery systems are prepared by a process including the step of forming or preparing a solution including a monomer, an initiator, and/or an active compound. In one embodiment, the process includes the step of applying or disposing the solution on a substrate, wherein the substrate includes at least one of a yarn, yarn precursor, thread, filament, fiber, textile, and/or another suitable substrate. In certain embodiments, the process includes the step of exposing the solution and the substrate to UV light to initiate polymerization and/or cross-linking of the solution. In one embodiment, the solution further includes an oligomer and/or any other suitable material or component.

Generally, fibers and yarns found in garments and textiles come from either natural sources (e.g., silk, cotton, etc.) or are synthetic (e.g., nylon, polyester, acetates, etc.). The selection of material type, fiber type, and mixture in a garment construction is operable to determine a number of factors for consumer benefit, use, and desire. Likewise, the method of construction (e.g., knitting, weaving, sewing, etc.) is also operable to determine at least a portion of the final attributes. Additionally, fibers, yarns, textiles, and finished garments are operable to be further processed or treated to create additional benefit.

One such method is fiber wrapping or covering. In certain embodiments, the yarn or substrate needs greater elasticity or stretch. Thus, in one embodiment, the yarn (i.e., a primary yarn) is operable to be plied or twisted with an air-covered yarn (i.e., a secondary yarn), such as spandex, to enable additional stretch of the yarn. In some embodiments, the secondary yarn, which wraps, coils, or covers the primary yarn, is hydrophobic or hydrophilic. Additionally, in one embodiment, the yarn is air-covered/air-intermingled (i.e., blowing air onto the yarn and adding a spandex core into the middle of the yarn). In one embodiment, the primary yarn is formed of nylon. These methods are useful for garments that need a lot of stretch, e.g., such as tights or leggings (or even the elastic portion at the top of a sock).

In one embodiment, the secondary yarn is wrapped or covered with filaments (e.g., nylon filaments) to make a fibril. A plurality of fibrils is combined to form a yarn. In one embodiment, the yarn is a multi-component, rope like structure with a plurality of interstitial spaces. The yarn is operable to be passed through a trough containing a polymer, oligomer, or monomer matrix, which fills the interstitial spaces. Additionally, some of the polymer, oligomer, or monomer matrix remains on a surface of the yarn. The wetted yarn is polymerized (e.g., via UV polymerization). The polymerized yarn hardens the polymer, oligomer, or monomer matrix. While the overall texture and elasticity of the polymerized yarn is changed relative to the original yarn, the polymerized yarn remains elastic and is operable to stretch.

Semi-finished garments and articles of wear are operable to be further treated through methods of finishing, treating, washing, dyeing, etc. For example, socks are operable to be boarded, steamed, and sized to fix the shape. Articles of wear are also often pre-washed to either remove excess dye or to soften the surface or appearance. Accordingly, in some embodiments, the coating and incorporated active needs to be resilient throughout the formation of a garment and/or post-treatments. Accordingly, in one embodiment, the coating is flexible with abrasion resistance, able to withstand sharp angles and points of frictional contact so as to avoid cracking or flaking, able to withstand processing temperatures, and/or has similar flexural properties to that of the fiber or yarn to move in concert with the fiber or yarn during deformation (e.g., stretching or folding).

Garments or wearable articles containing active fibers are operable be tested in various ways for efficacy, e.g., measurement of active release and/or resistance to active loss by use, wear, application, and/or laundering are operable to be performed. For example, a solvent extraction method on a basic, coated yarn or final garment is operable to be conducted. While water or synthetic sebum/sweat are operable to be used, so too can basic solvents that accelerate potential active release. Accelerating release is operable to allow for faster, correlated analysis to be done and is operable to be geared towards working with a solvent that solubilizes the active. The extract arising from such extractions is then operable to be characterized, e.g., by HPLC to determine the quantity of active released based on the time and conditions of extraction. As to studying resistance to laundering, fibers, yarns, and garments are operable to be washed under controlled conditions and time (e.g., cycles) followed by an extraction as discussed above. Comparison of extraction data before and after laundering is operable to be used to determine the quantity of active lost.

The selection of monomers in a curable system or the selection of a pre-made polymer coating is operable to be varied to enhance necessary functions including, but not limited to: coupling to the substrate (e.g., yarn, fiber, etc.); compatibility with and/or the release profile of the active; physical and/or mechanical properties to aid garment creation (e.g., knitting); and/or to manage the tactile and other aesthetics of the yarns, fibers, or other substrates. Additional information about monomers, oligomers, and/or polymers compatible with the present invention is in U.S. Patent Publication No. 20200390720, which is incorporated herein by reference in its entirety.

Certain embodiments of the disclosure rely substantially, or even entirely, on the release characteristics of the polymer matrix that is in direct contact with the active. As described above, the embodiments described herein are operable to include solid active (e.g., crystalline active, active powders, etc.) dispersed in a polymeric matrix, and configured such that the egress of active from the matrix is substantially limited by the appropriate shape and coating of the polymeric matrix, so as to achieve a near-constant rate of active release over an extended period of time.

Monomers are operable to be selected based meeting a variety of criteria including, but not limited to: compatibility with the active, control of hydrophobicity, specific ability to cross-link via multi-functionality, viscosity for pumping coating, wettability and potential adhesion to the yarn or substrate, etc. In one embodiment, the monomer blend also includes a free radical initiator and/or a UV-activated initiator to allow for UV-curing of the coating. Such initiators are aligned with specific light source wavelengths. The oligomer/monomer components are often warmed (e.g., between about 25° C. and about 50° C.) to enhance mixing followed by initiator and then incorporation of the actives to form a stable solution or dispersion.

The various embodiments of the present disclosure are operable to include or utilize polymers, prepared in situ on the yarn, fiber, or substrate or coated and/or applied from solution, thus forming a functional coating, which serves as a protective matrix for beneficial actives and in which, in turn, controls their delivery. In certain embodiments, the polymer is operable to be hydrophobic and/or cross-linked. Cross-linking (also referred to as “curing,” “vulcanizing,” and “thermosetting”) applied to a dispersion or suspension of active particles in a polymer, oligomer, or monomer matrix, commercial coating or adhesive, chemically reactive linear polymer, etc.—is operable to be employed by the various embodiments of the present disclosure for preparing yarns, filaments, textiles, and fabrics, providing for protection of the beneficial active against excessive loss during laundering, as well as against a wide range of chemical degradation reactions including hydrolysis, oxidation, acid/base-catalyzed reactions, etc. The polymer matrices are operable to be formed from various polymer- or oligomer-based systems, including commercially available elastomeric adhesives, glues, coatings, caulks, sealants, casting materials, curable monomers and cross-linking systems. The various embodiments of the present disclosure are also operable to be formed from one or more monomers and or polymers in any combination.

In some embodiments, after the polymerization of the dispersion or suspension of active particles in a polymer, oligomer, or monomer matrix on the yarn or textile, the yarn or textile is operable to be rewound into a bundle such as a ball, skein, hank, cone, or cake for later use in the knitting of garments.

In specific embodiments, the functional coating demonstrates that the polymers are operable to be used as a vehicle to load one or more actives into and/or onto the a yarn, yarn precursor, thread, filament, fiber, textile, or other substrate and/or immobilize the one or more actives in and/or on the yarn, yarn precursor, thread, filament, fiber, textile, or other substrate. For example, in particular embodiments, one or more actives is combined with a polymer or mixed into a curable monomer system to form a mixture or solution, which is applied to a yarn, yarn precursor, thread, filament, fiber, textile, or substrate. In some embodiments, the polymerization occurs in the presence of the dispersed, dissolved, or suspended active particles, resulting in a configuration in which local stresses and strains on the polymer associated with “forcing” solid active particles into an already-cross-linked polymer are minimized or eliminated. In specific embodiments, the polymer is operable to be cross-linked. Such strains, at least at high active loadings, are operable to lead to higher permeability and loss of active-protecting effect. Entry of solid active particles (e.g., crystals) into, or formation inside, a previously cross-linked polymeric core is also operable to cause distortion of the structure, leaving the active accessible when the purpose of encapsulation is to make it inaccessible. In other embodiments, however, all or a portion of the cross-linking occurs prior to introduction of the active.

Some of the embodiments disclosed herein include cross-linking so as to lock, hold, or otherwise temporarily retain active particles or their conjugates in place and protect them from degradation and premature loss, particularly in the face of stress conditions, such as those encountered in laundering.

### EXAMPLES

The following examples illustrate the present invention but are not to be construed as limiting the invention.

### Example 1

The purpose of this first experiment was to determine if a coating could be found that is operable to wet the surface of the silicone used to form the polymeric or elastomeric matrix in some embodiments and examples herein. Specifically, a Room Temperature Vulcanizing (RTV) silicone polymer sold as Novagard 200-260 was selected for application because of its low viscosity (approximately 400 centipoise), which allowed for both simple processing and good uptake. Novagard 200-260 is 100% silicone and begins cross-linking upon contact with air; the skin-over time is listed as 35 minutes.

Silicone is, in the liquid state, a fluid that wets and spreads over just about any other solid material. This is operable to be favorable for application to an existing yarn or other substrate material.

However, for essentially the same reasons, cross-linked silicone is a very low surface energy material that can be extremely difficult to coat uniformly—coatings tend to “bead up” like rain on a freshly-waxed windshield. Simply phrased, silicones are typically spread on other materials, and other materials do not typically spread on silicone. For example, those who work with paints generally consider silicone to be an “unpaintable” material.

Therefore, a wide range of commercially available coatings, both sprays and brush-ons, were tested for their ability to spread atop cured Novagard 200-260. Films of the RTV were poured onto a piece of cardboard and allowed to cure, after which the various coatings were applied as per instructions and normal usage. A 10×-magnification eye loop was used to examine the coatings, most of which were readily seen to be beaded up and not continuous. The non-viable coatings included cyanoacrylate (“super glue”), epoxy, natural rubber, acrylated silicone, various acrylics, and a number of adhesives that did not provide the chemical composition.

Two coatings were found to provide a continuous, smooth coating:

1. A zinc-based spray-applied coating marketed by Clearco Corp., under the product name “High Performance Zinc Spray”; the spray forms a coating that is over 90% zinc oxide; and

2. Vinyl coatings from several manufacturers, including Rust-Oleum® Specialty Vinyl Spray, which is the vinyl coating used in some other Examples below.

Both types of coatings surprisingly spread on the Novagard 200-260 silicone so as to coat the silicone surface uniformly and continuously when applied as sprays. With the vinyl coating, a uniform coating was also achieved when sprayed into a container and then applied as a brush-on liquid.

### Example 2

Usnic acid is a naturally derived compound (from lichens) that functions as an analgesic, antiviral, antimitotic, and anti-inflammatory active, and has been used for its apparent activity in helping people lose weight. This active, obtained as a fine powder, was suspended at a loading of 2% by weight (20 mg/gm) in a sample of Novagard 200-260 RTV silicone polymer. Usnic acid is a good active for release experiments because it is strongly absorbing at wavelengths around 300 nanometers, in addition to being very useful for personal health.

The suspension of usnic acid in Novagard 200-260 was then applied to 30-weight cotton thread (mercerized, 100% cotton), by passing the cotton through the usnic-in-RTV suspension over a length of about 10 inches. Weighing identical lengths before and after treatment showed that the thread doubled in weight, i.e., that the increase in weight per unit length was about 100%, or (3=1.0.

It was found that if yarn segments contact each other during the process of curing (cross-linking), then they become difficult to separate, making retrieval of usable yarn a very difficult process. Therefore, a special apparatus was designed that collected the freshly treated yarn in such a way that it isolated each segment of yarn from the rest of the yarn and from any other material, except for small (approximately 0.5 inch) contact points every 9 inches. At these contact points, the treated yarn was resting against a screw-threaded steel rod, six of which were aligned vertically in a hexagon arrangement of diameter 18 inches, and the cured yarn did not stick strongly to the metal. Briefly, as a carousel containing these six 3-foot-long rods was spun by a motor drive, the freshly treated yarn was directed into the screw-threads (13 per inch) on the steel rods, dropping down one screw-thread per carousel revolution. This ensured that approximately 95% of the treated yarn was free from contact with anything except air during the time that the treated fluid was drying and/or curing.

After curing at room temperature for 24 hours, a zinc oxide-based coating was applied from a spray can to the treated (e.g., with the active composition) and cured thread. The coating used was the “High Performance Zinc Spray” described in Example 1. Some of the treated yarn was intermittently coated, as per certain embodiments of the present disclosure. Other portions of the treated yarn were fully coated, having 0% open area for testing the coating properties. A fully coated, as opposed to intermittently coated, yarn should exhibit minimal release at the appropriate timescale. This was tested in the next example.

### Example 3

The fully coated yarn of Example 2 was tested for the occlusiveness of the coating, using conditions that are extreme for a coating. The yarn was placed in an organic solvent, 1-pentanol, that not only solubilizes (dissolves) the active usnic acid, but does so very quickly due to low solvent viscosity and MW, and also tends to swell or even solubilize just about any material it comes into contact with.

Portions of yarn from Example 2, of uncrimped length 44 centimeters, and with various coating extents, were immersed in 20 milliliters of 1-pentanol, and samples at 0, 30, 120 minutes and 24 hours were analyzed for absorbance at 290 nanometers, near the major absorbance peak of usnic acid. Five samples were analyzed, and it should be noted that the variability of active loading along the yarn was very high, as this is a sensitive parameter to control in the embodiments described herein:

Sample A: a “primer” coating applied at 100%;

Sample B: uncoated control #1;

Sample C: Clearco Zinc/binder-based spray applied at 100%;

Sample D: uncoated control #2;

Sample E: un-imbibed/untreated control, no active.

Table 2 shows the absorbance, in milli-Absorbance units, at the 30- and 120-minute and 24-hour time points:

TABLE 2

| ID | Description | 30 min | 120 min | 24 hours |
|---|---|---|---|---|
| A | 100% primer | 18 | 60 | 74 |
| B | uncoated | 18 | 35 | 69 |
| C | 100% zinc oxide coat | 2 | 0 | 17 |
| D | uncoated | 8 | 46 | 47 |
| E | control, no active | 0 | 9 | 21 |

The data in Table 2 show, first of all, that the zinc oxide coated sample released far less than uncoated or “primer-coated” yarn of the same structure before coating.

A more detailed analysis may be justified. The data indicate that something other than usnic acid was solubilized from the matrix and contributed to the absorbance. Sample E suggests that the absorbance at 24 hours had a non-usnic contribution of approximately 20 milli-Absorbance units. With this approximation, Sample C was seen to be non-releasing, in this experiment. This is in sharp contrast with the ineffective coating of Sample A, which had absorbances comparable to the uncoated controls.

The availability of coatings such as the Clearco Zinc-based coating that have the ability to strongly inhibit—if not reduce to negligible levels—the release of active from a cross-linked silicone is a surprising result particularly for those who subscribe to the prevalent notion that silicone is an “uncoatable” or “unpaintable” material.

This Example also demonstrates that coatings exist which are able to occlude an active-loaded, silicone-treated yarn against active release, at 100% coating.

### Example 4

An 80 milligram piece of cotton thread treated with the same usnic acid/Novagard 200-260 suspension described above was cut into two 40 milligram pieces. One of the pieces was then fully coated (100%, with 0% open) with a coating by Valspar called “Rustoleum Vinyl.” Absorbances at 290 nanometers were taken after 22 hours of immersion in 20 milliliters of pentanol. The absorbances were as follows (a 10-fold dilution was used to keep the absorbance in the range of the instrument, and then factored back in for the final result): Uncoated: 6.780; Coated (with Vinyl Rustoleum): 2.230.

Thus, this Example also showed strong retention of active even when the entire thread is immersed in a solvent liquid (pentanol).

### Example 5

This Example demonstrated zero-order release using a partly but dominantly coated silicone core loaded with usnic acid as active. Samples were prepared by taking a fine, hollow tube of nylon, and loading it with a suspension of usnic acid in a silicone RTV known as “Silicone Ultra,” made by White Lightning, which is 100% silicone.

Sample “A”: 0.117 grams usnic acid+0.953 grams Silicone Ultra, all of which was loaded into the nylon tubing.

Sample “B”: 0.101 grams usnic+1.023 grams Silicone Ultra, 0.593 grams of which was loaded into the tubing.

While approximately 99% of the usnic/silicone was surrounded by (i.e., coated by) the nylon tubing, after curing approximately 1% of the usnic/silicone protruded out of the tubing and was thus uncoated. The uncoated end of each sample was then immersed in 100 milliliters of a solvent mix with the following composition: 56.1% acetonitrile (ACN), 17.5% water, 14.2% tert-butyl acetate, and 12.2% tetrahydrofuran (THF). The two solutions were then analyzed periodically over the next 2 months for absorbance at 310 nanometers, which is determined almost entirely by the concentration of usnic acid in the solvent mix. The solvent mix was stirred gently before each sampling.

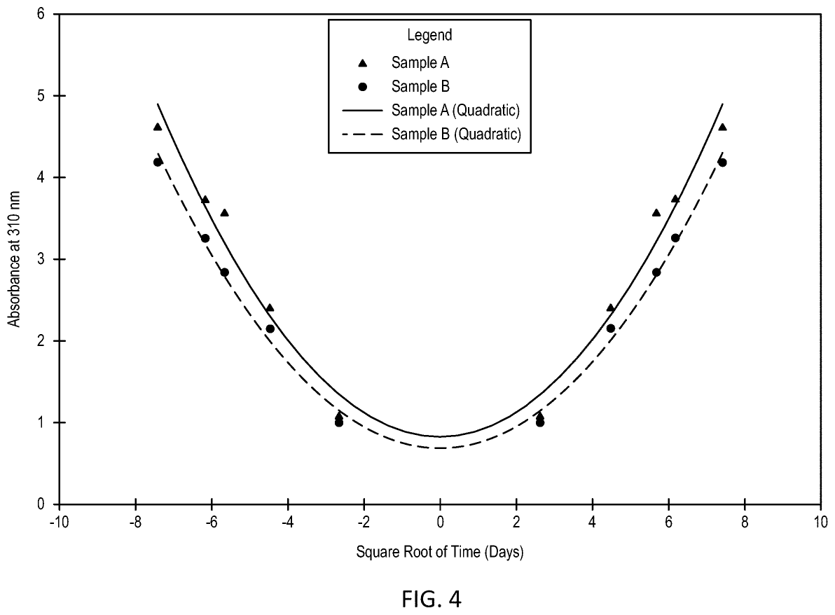

FIG. 4 shows the UV-absorbances at 310 nanometers plotted against the square root of time in days, for both Samples A and B. Diffusion-limited processes yield a cumulative release curve that varies as the square root of time. If that were the case here, the plots in FIG. 4 would be straight lines. However, if a best-fit linear fit is performed on the data, the Y-intercept—which is the concentration at time zero calculated using the linear fit—is strongly negative, namely −0.801. Not only is this not possible and far outside the precision of this experiment, but also the quadratic fits shown fit the two data sets (reflected across the Y-axis so as to force a purely quadratic fit, with no linear term) to very high regression coefficients, namely R=0.980 and 0.994. In addition, the Y-intercept (time zero) is positive, and in fact the value of 0.5 is in agreement with background absorbances in similar experiments. A quadratic fit, when plotting the absorbance against the square root of time, means that the absorbance varies linearly with time. And since the concentration is related to the absorbance by a constant (the molar absorptivity), this example shows that zero-order release kinetics—where the cumulative amount of active released is proportional to the time, making the release rate a constant—is indeed observed with the partly but dominantly coated architecture.

Another similar sample, but with a (soft-segment) polyurethane as a polymeric or elastomeric matrix material, and a concentration of usnic acid that is only half the above case, showed a release rate that was approximately the same as that of this silicone-based material.

### Example 6

Using the apparatus described in Example 3, yarns as per embodiments of the present disclosure were prepared using several chemistries. In each case at least 100 yards, and in most cases over 300 yards, were produced. The chemistries are summarized in Table 3. In each case, the active was loaded to a level of 1% in a polymeric or elastomeric matrix. The “zinc oxide” coating in Table 3 refers to the “High Performance Zinc Spray” from Clearco discussed above.

TABLE 3
(*Kraton IR401)

| Active compound | Polymeric matrix | Coating | Substrate yarn |
|---|---|---|---|
| Hydrocortisone | Polyisoprene | Zinc oxide | 1/150/34 polyester |
| | emulsion* | | |
| Usnic acid | Novagard | Rustoleum | 30-wt cotton |
| | 200-260 | Vinyl | |
| Pyrithione zinc | Novagard | Rustoleum | 1/150/34 polyester |
| | 200-260 | Vinyl | |
| Retinoic acid | Novagard | Zinc oxide | 1/150/34 polyester |
| | 200-260 | | |
| CoEnzyme Q10 | Novagard | Zinc oxide | 30-wt cotton |
| | 200-260 | | |
| Curcumin | Polyisoprene | Zinc oxide | 1/150/34 polyester |
| | emulsion* | | |
| Curcumin | Novagard | Zinc oxide | 1/150/34 polyester |
| | 200-260 | | |
| Arecoline | Novagard | Zinc oxide | 1/150/34 polyester |
| | 200-260 | | |

In order to cross-link the polyisoprene in the two cases above (first and sixth rows), the yarn was placed in an oven at 300° F. for one hour.

### Example 7

An uncoated, treated yarn, with polyester substrate yarn treated with a 10% by weight suspension of arecoline hydrobromide in Novagard 200-260, was woven into a small piece of fabric made 100% from that yarn. This was then tested in a Franz cell apparatus (Zyleris Pharmatech) for its ability to deliver the active (arecoline) transdermally. Another portion of the treated yarn was intermittently coated as per an embodiment of the disclosure (see the last row of Table 3), but for demonstrating transdermal delivery it was reasoned that uncoated was best.

One skilled in the art will be familiar with the design of a Franz cell. The test article, in this case the aforementioned arecoline-loaded fabric, was placed atop a small piece of freshly excised skin, in this case from a pig's ear; below the skin was a reservoir containing bovine serum albumin buffer to simulate blood plasma. In order to reach the reservoir, the active had to diffuse transdermally across the layer of skin. Three such Franz cells were used so that the experiment was done in triplicate. A small aliquot was drawn from each reservoir at the 24-hour point and tested for arecoline as now described.

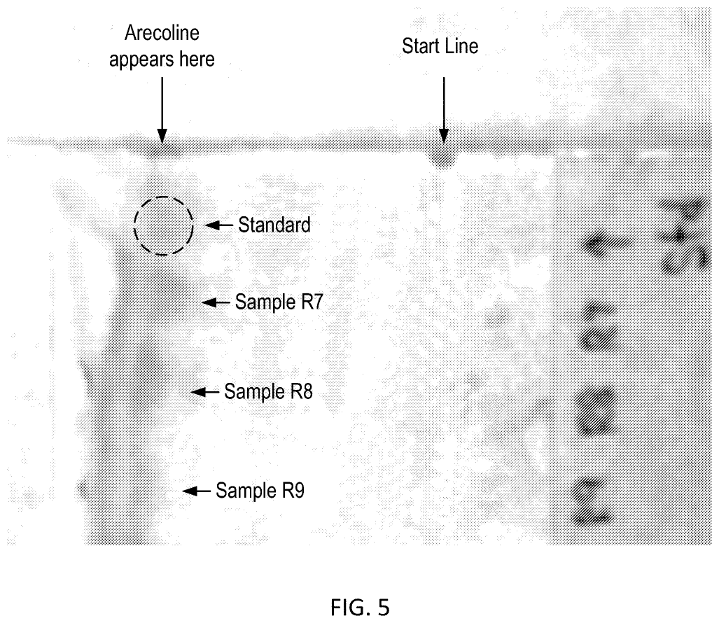

A reference arecoline solution was prepared by dissolving approximately 3 milligrams arecoline hydrobromide (ScienceLab.com) in approximately 0.5 milliliters of bovine serum albumin buffer, for an approximate concentration of about 6 milligrams/milliliter. The reference solution was spotted alongside all three samples (labelled R7, R8 and R9) on a TLC plate at the origin. After driving out the water from the spots with heat, the spotted TLC plate was allowed to cool, then developed in 100% methanol inside a developing tank. After development, spots were visualized after 1) dipping the plate in 0.02 M aqueous copper nitrate solution, 2) heating on a hotplate, 3) allowing it to cool, 4) dipping in 0.05 M aqueous potassium iodide, and 5) heating on a hotplate. The reference solution yielded a faint brown spot running just behind the solvent front. All three sample solutions yielded a brown spot of the same order of intensity as that from the 6 milligram/milliliter reference solution, and at the same retention factor as the brown spot from the reference solution. See FIG. 5 for a photograph of the resulting TLC plate.

The approximate retention factor was 0.88. Based on the observation of equal, or even greater, spot intensity for the samples as compared to the reference, the concentration of arecoline in the reservoirs was on the order of 5 milligrams/milliliters. Thus, the treated yarn is effective at transdermal delivery of arecoline, according to this standardized pig ear skin Franz cell model.

### Example 8

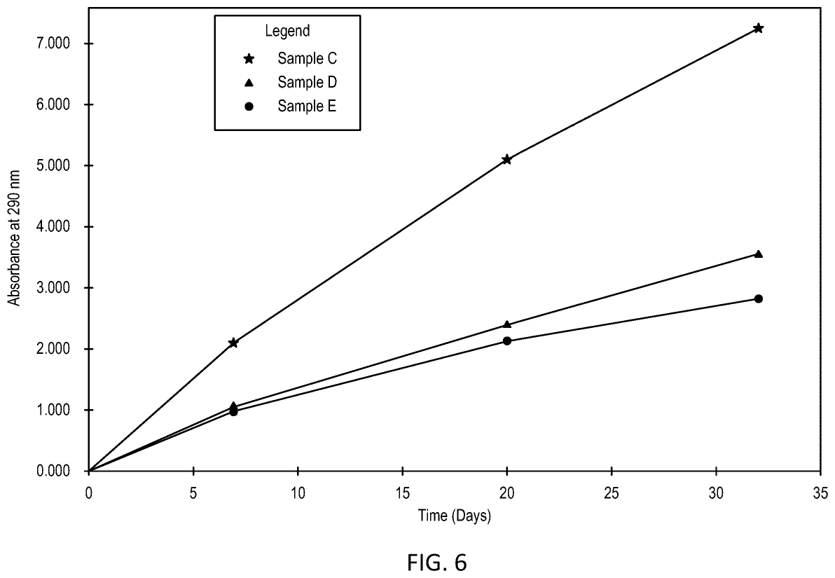

Exemplary drug release profiles according to embodiments of the present disclosure are depicted in FIGS. 6-8. These drug-release profiles were measured from intermittently coated yarns and are consistent with near-zero order release kinetics.

Referring first to FIG. 6, these results were obtained from UV-Vis spectroscopy and represent the release of the naturally occurring antifungal and antimicrobial compound usnic acid. Samples C-E each contained one yard of 30-weight cotton yarn treated with a matrix polymer containing dispersed usnic acid. After curing the matrix polymer, each of Samples C-E were intermittently spray coated (approximately 80% coated) with an aerosol product marketed as Rustoleum Vinyl. Sample C contained a polyurethane matrix polymer (Rovene 4021), and the amount of matrix polymer incorporated onto and/or into the yarn approximately doubled the weight of the yarn, i.e., a weight increase on the order of 100%. Samples D and E each contained a polysiloxane matrix polymer (Novagard 200-260), and the amount of matrix polymer incorporated onto and/or into the yarn approximately doubled the weight of the yarn, i.e., a weight increase on the order of 100%. The samples were then placed in a pentanol solution and gently rocked, during which the release of usnic acid was measured as the absorbance at 290 nanometers, the results of which demonstrated a near-zero order release as depicted in FIG. 6.

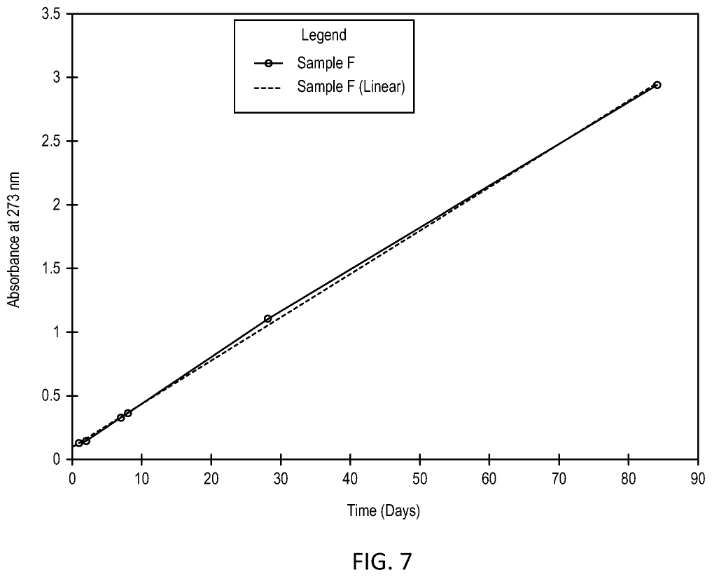

FIG. 7 depicts near-zero order release of terbinafine hydrochloride. Sample F contained one yard of polyester yarn (150 Denier) treated with a matrix polymer of polyurethane (Rovene 4021) containing dispersed terbinafine hydrochloride. The amount of matrix polymer incorporated onto and/or into the yarn approximately doubled the weight of the yarn, i.e., a weight increase on the order of 100%. A polyurethane coating, marketed under the name “ZAR Exterior Polyurethane,” was intermittently applied to achieve a coating on approximately 90% of the yarn. The release of terbinafine hydrochloride into water was measured as the absorbance at 273 nanometers, the results of which are depicted in FIG. 7, which also includes a linear fit of the data points.

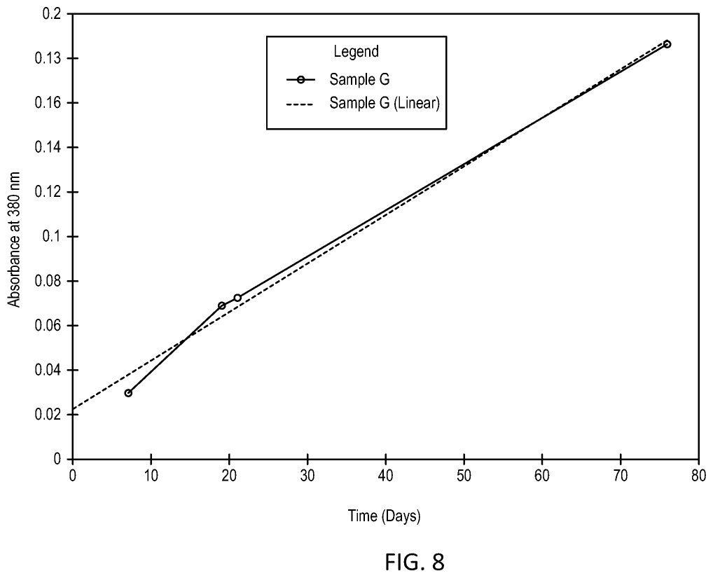

FIG. 8 depicts the near-zero order release over 3 months of the antispasmotic drug dantrolene sodium from a one-yard portion (Sample G) of intermittently coated yarn (150 Denier polyester yarn) as per the disclosure, releasing into a weakly buffered aqueous solution at pH approximately 11.0, with absorbance measured at 380 nanometers, a known absorbance peak of aqueous dantrolene sodium. FIG. 8 also includes both the absorbance measurements and a linear fit of the data points. Novagard 200-260 RTV was used as the polymeric matrix in which the dantrolene was dispersed. The amount of matrix polymer incorporated onto and/or into the yarn approximately doubled the weight of the yarn, i.e., a weight increase on the order of 100%. The release kinetics of this strongly absorbing (and thus accurately measured) drug is very close to perfect zero-order, constant rate of release. The coating applied was a “hard” polyurethane coating supplied as an aqueous dispersion purchased from Alberdingk-Boley under the product designation “Aliphatic Polyurethane Dispersion U-933.” The coating was applied by a brush-painting operation, performed by a professional artist instructed to coat 10 centimeter-wide stripes separated by unpainted (uncoated) stripes of approximately 2.5 centimeters (resulting in an approximately 80% coated yarn); the stripes were vertical on a vertically oriented, batch-mode accumulator.

### Example 9

This example demonstrates exemplary coating methods according to embodiments of the present disclosure. A spray-coating was conducted using a model AA10000JJAU-03 spray gun, a PFJ2050 fluid cap, and a PAJ45350-40-SS air cap (each from Spraying Systems Co.). After each segment of testing the spray tip was submerged in water and fired several times in order to keep the acrylic from hardening.

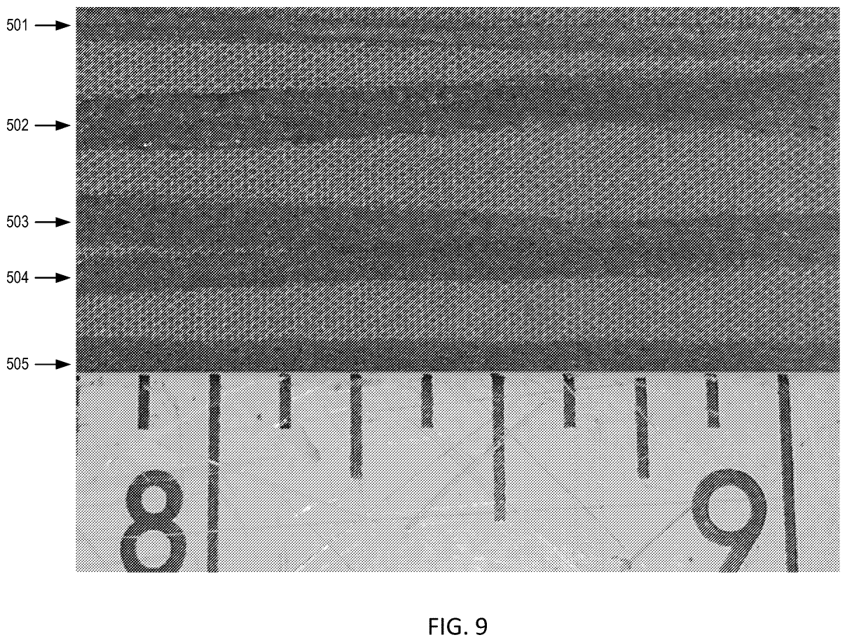

A bulked or textured 150 Denier yarn was first treated with Novagard 200-265 “fast-cure” RTV, after which the bulk and/or texture was maintained. After drying, approximately 3 yards of the treated yarn was wrapped around a 5-inch open frame, and the spray gun loaded with Alberdingk AC2523 self-cross-linking acrylic coating mixed with a green food coloring to aid in visualizing the coating. The loaded spray system was pulsed 15 times at 20 milliseconds per pulse, spraying at a nozzle-to-yarn distance of approximately 5 inches. This provided a rather uniform coating, and as seen in the close-up photograph in FIG. 9, the bulk or texture of the yarn was maintained—in other words, each individual fibril was coated separately. This offers many advantages over other coating processes that “glue” the fibrils together resulting in a “flat” yarn, such as increased comfort, circumvention of the need to ply the yarn, increased surface area of skin contact, and compatibility with standard yarn and textile processes. As is also seen in FIG. 9, a ruler was placed alongside yarn samples (501, 502, 503, 504, 505) to demonstrate that the bulk or texture was maintained, since a flat yarn of this Denier would be less than 1/64th of an inch in width, whereas this yarn's bulk or texture provided an expanded width that was over 1/16th of an inch. In another similar coating method, 1.5-inch wide strips of a soft foam were glued to a drum 7 inches in diameter, leaving ¼-inch gaps between them.

### Example 10

Experience with water-borne coatings, such as Alberdingk acrylic dispersion “AC 2523,” indicated that texture can be difficult to maintain upon coating a bulked or textured yarn with a formulation that contains 20% or more water, whether the yarn had been treated with RTV or not. Even when tension on the yarn was kept below 10 grams, bulk or texture was lost after the coating had dried/cured, resulting in a “flat” yarn. This Example used a water-free, solvent-free coating to test whether bulk or texture could be maintained.

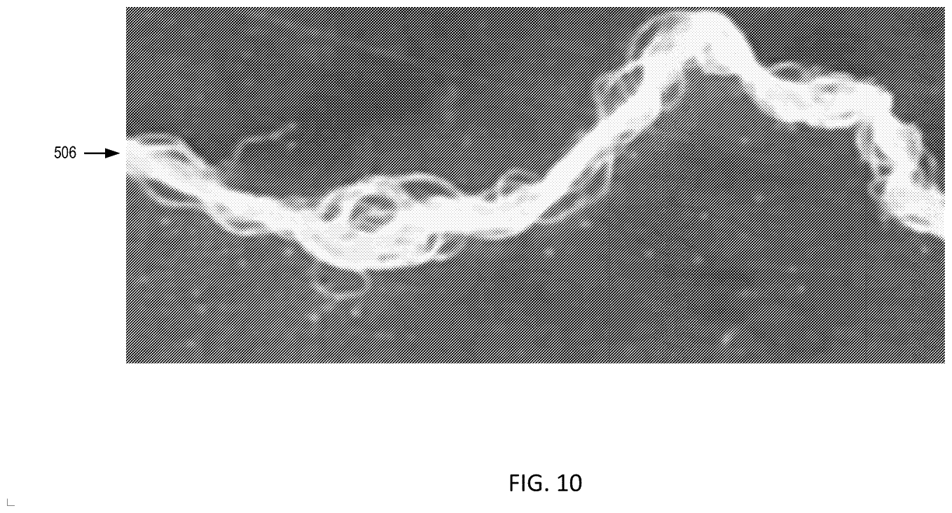

A cyanoacrylate adhesive, Gorilla superglue, was applied to coat a yarn which had previously been treated with a dispersion of aspirin powder (25% by weight) in Novagard 200-265 ultralow viscosity RTV. The treated and cured yarn, still bulked and texturized, and of measured Denier of approximately 90 D, was then passed through a small container of Gorilla superglue, with the residence length being approximately 2 millimeters and the residence time on the order of 10 milliseconds. Tension in the line was not measured but was high, well in excess of 10 grams. The Denier increased to over 300 D, as the yarn picked up a very large amount of superglue. Nevertheless, the final yarn, after the superglue had cured, was still bulked or texturized. FIG. 10 shows a photograph of the final yarn 506 of the invention after treatment with RTV/aspirin and subsequent coating with the cyanoacrylate. While the photograph does not capture all of the detailed structure, the bulk and texture are evident.

### Example 11

This example provides an exemplary method for large-scale yarn production. Tests in the inventors' lab have demonstrated the feasibility of each step discussed here, and one skilled in the art will understand the methods described. Bulked or textured yarn coming off a creel will first pass through a reservoir of RTV or other matrix source, which has a low enough viscosity that it is covers each fibril of the yarn; if the viscosity is too high, then the Deborah number of the treatment may be too high to provide a contiguous film of matrix. At a typical yarn speed on the order of 10 meters per second, the yarn passes through a chamber or sack containing a thickness of matrix on the order of ¼ inch, making a residence time of ¼ inch/10 meters/second or roughly 1 millisecond. At least in the case of a silicone-based matrix, results have consistently shown that this is sufficient time to leave a contiguous film of silicone on each fibril of the yarn (for textured polyester and nylon yarns), and the Denier increases by approximately 50%. For cases where a larger loading of active is required, the residence time is operable to be increased to several milliseconds. However, if the Denier increases by more than 100% (i.e., more than doubles in weight per unit length), there is increasing risk that the yarn will go flat and lose texture, which is generally undesirable. In one embodiment, it is advantageous to maintain an inert, dry atmosphere at the chamber to limit or eliminate any premature curing of the RTV.

After passing through the chamber and applying the RTV (or other matrix source), the RTV should be substantially cured before moving on to the coating stage, otherwise the low surface energy of the matrix is operable to promote migration of the wet RTV over the intended coating. Strong ultraviolet light is operable to cure some RTVs (such as Novagard 200-260) in a few seconds, though this presents some costs and exposure hazards. Warm, humid air is operable to be used to trigger or initiate the cross-linking reaction, which in the case of Novagard 200-265 is substantially complete in 3 to 5 minutes. A single-end or multiple-end slasher is then used to temporarily wind the treated, drying yarn, moving it along slowly such that during the 3 to 5 minute curing time, only a small fraction (less than 10%) of the drying yarn contacts any solid and yarn-yarn contacts are avoided. At a production speed of about 10 meters per second, the slasher needs to hold about 2,500 yards of yarn per end in order to provide adequate drying time before moving on to the next step which involves contacting a solid. Yarns should be spaced approximately 1/16 inch apart. In one embodiment, a multi-end slasher is desirable, particularly since most commercial slashers space the yarns at considerably higher spacings than 1/16 inch, leaving ample space for multiple yarn ends while still avoiding or at least minimizing contact between adjacent winds.

Coming off the slasher, the now substantially dried yarn passes around a drum which is rotating slightly faster than a second drum on the exit side of the coating chamber; this “relaxes” the yarn so that in the coating process, the fibrils making up the yarn are “bulked,” or substantially separated from each other in a more open configuration. Other methods, such as invoking accumulators, are known in the art for relaxing yarn during continuous-mode production steps. The treatment step is generally quite forgiving of tension—flattening from over-applying the RTV rather than from tension—and so it is generally not a problem if the faster-rotating drum is controlling the speed of passage through the application chamber (e.g., applying the at least one active composition).

After passing around the first (faster-spinning) drum, the yarn enters the coating chamber which, depending on the plant/worker conditions and details of the coating chemistry, might benefit from an enclosure, with very small openings for the entry and exit of the yarn. The (now-relaxed) yarn then passes in front of an array of spray nozzles, e.g., two nozzles for the “left” and “right” sides of the yarn. While a pulsing of the spray gun is possible, intermittency of the coating is more sharply defined if a mask is used. Thus, a belt with openings cut into it is driven around a system of pulleys such that the velocity of the belt matches that of the yarn (about 10 meters per second) over the region where the belt comes between the nozzle and the yarn. For example, in one embodiment, two belts are necessary for a two-nozzle system. Between the openings in the belt/mask are solid regions blocking or diverting the spray so as to leave uncoated segments on the yarn of the desired length; in general, these will constitute on the order of 10% of the yarn, since 90% will be coated, so that the wasting of this “blocked” fraction of the spray is small. Alternatively, this mask is operable to be timed with a pulsing of the spray, eliminating most of the wastage due to blocking while still allowing for a crisp, well-defined intermittent pattern on the yarn.

Multiple ends entering the coating chamber provide not only a higher production rate, but also more efficient use of coating (which may be difficult or expensive to recirculate). Spray nozzles capable of restricting the spray to 1/16 inch width are difficult to come by, so if, say, 12 yarns are spaced 1/16 inch apart at the point of coating application, this three-fourths inch is much closer to the typical width of a spray pattern.

In one embodiment, after passing from the coating chamber (and enclosure, if present), the yarn needs to be stored on a second slasher, if the coating takes minutes to dry. However, tests with the acrylic coating Alberdingk AC 2523, for example, have shown that yarn is operable to be wound onto a cone directly from the coating chamber, skipping any slasher or accumulator, provided that the Denier of the yarn is below about 200 (yarn thicker than this is more prone to sticking to itself), particularly if a few tens of feet are traversed by the yarn between the coating and the cone. Drying methods, as simple as blowing air onto the yarn, are operable to be applied during the traversal of this distance.

Many post-processing steps are then operable to be applied as needed. If conditions are such that the yarn is operable to benefit from plying with one or more other yarns, then plying is operable to be used to alter the “hand,” color, elongation, or processibility, or to combine properties of two or more yarns.

### Example 12

This Example provides exemplary release tests on fabrics from yarns of the disclosure. These release measurements were performed on small swatches of fabric produced from yarn of the disclosure, and washed or otherwise stressed before release testing. Release was used to quantify how much active is retained intact after the stress. No coating was necessary for this test. Retention of active through stresses such as washes and scouring is a key distinguishing benefit of yarns of this disclosure and enables medicated fabrics that are operable to be applied in a wide range of applications that demand washability in order to be commercially viable.

Aspirin (acetylsalicylic acid) was ground to a fine powder and dispersed at 25% wt/wt in Novagard 200-260. This was applied to a texturized 70 Denier nylon yarn, then plied with a textured 70 Denier nylon yarn. The yarn was then knitted into several sleeves. In an informal test, wearing a sleeve was sufficient to provide significant analgesia when worn on the elbow or knee.

Washing with hot detergent water was then done once (on swatch #2), 5 times (swatch #3), or 10 times (swatch #4). Swatch #1 was the control with zero washings. Each swatch, a fixed 1-inch square, was placed in water for release, and after two days the absorbance measured at 280 nanometers.

TABLE 4

| | Control | | | | |
|---|---|---|---|---|---|
| | (0 washes) | 1 wash | 5 washes | 10 washes | Dyed |
| Absorbance | 0.097 | 0.077 | 0.038 | 0.045 | 0.051 |
| (at 280 nm) | | | | | |

These data show retention of about 45% of the active after 10 washings.

Another sleeve was dyed using a standard dyeing process, which included scouring at the upper limit of the normal range of temperatures in dyeing, namely 220° F. for 45 minutes. This result is shown in the last column of the above table. Over 50% potency was retained through this intense process.

### Example 13

Allantoin, which is frequently used as an active in skin creams, was dispersed in RTV at 25%, and this matrix applied to a 40 Denier texturized nylon yarn. Some of this was plied with a 40 Denier nylon yarn. Another portion was air-covered with a spandex yarn. Testing revealed a skin-soothing, softening effect when used, due at least in part to a moisturizing effect.

### Example 14

Other actives were incorporated into or onto yarns in accordance with the present disclosure, including caffeine, ibuprofen, acetaminophen, cetyl palmitate, capsaicin, and menthol. In each instance, the active was successfully incorporated into or onto a yarn substrate with a polymer matrix.

### Example 15

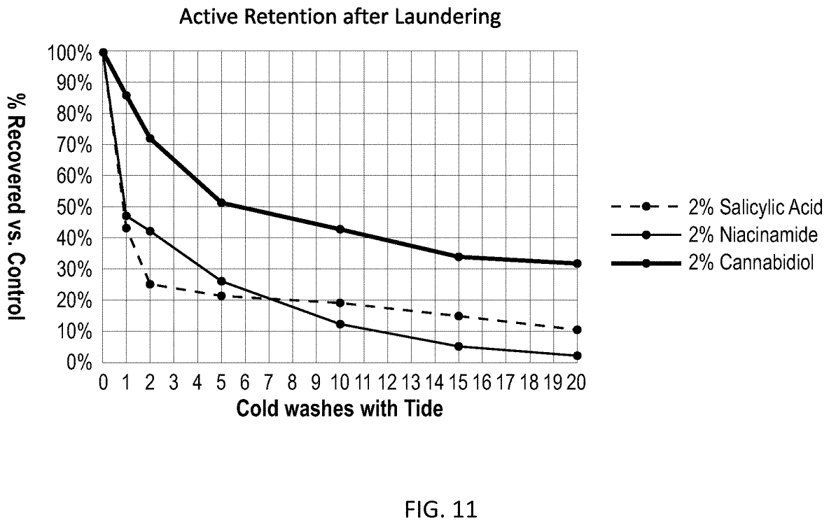

90-meter coated yarn samples were laundered for approximately 40 minutes with a cold wash using TIDE® detergent. Yarn samples were removed at various points, air-dried, and remaining active was extracted with a solvent. The resulting extract was analyzed by HPLC to determine the total active present in the yarn. This was plotted as a percentage of the active extracted from an unwashed control sample (see FIG. 11).

### Example 16

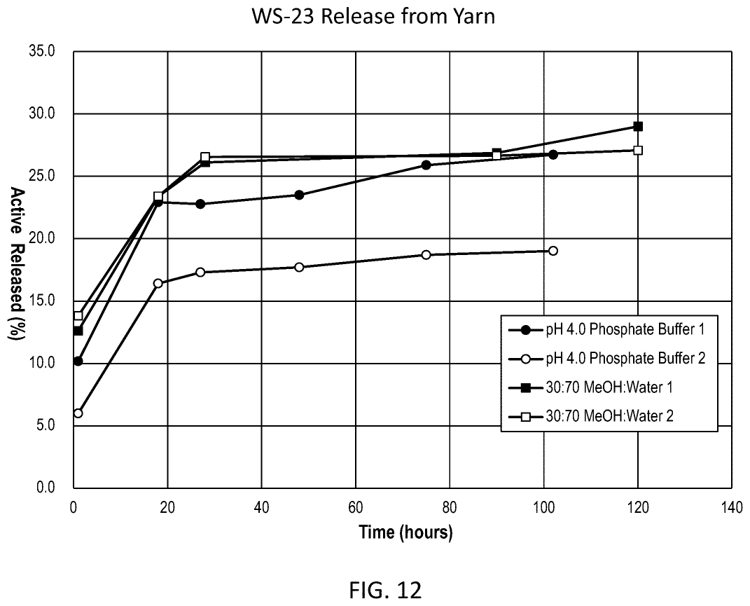

90-meter coated yarn samples containing 15% 2-isopropyl-N,2,3-trimethylbutyramide (WS-23) were submerged in two different solvent systems to release active. Solvent was sampled periodically and analyzed by gas chromatography (GC). Release was plotted as a percentage of total active present in yarn (see FIG. 12).

### Example 17

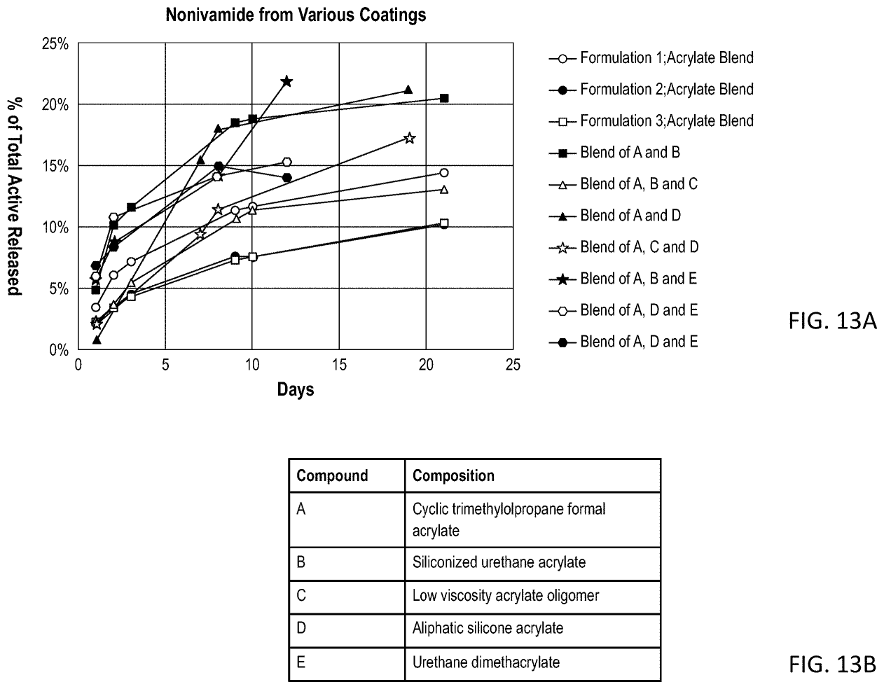

Various coating formulations were loaded with 5% nonivamide. 2.0 gram disks of each coating were cured by UV under N₂. These samples were then submerged in 100 mL of a 30:70 MeOH:water solvent system. This solution was sampled over a period of up to 21 days with volume replacement after each sampling. Solutions were analyzed by HPLC and plotted as a percentage of total nonivamide present in each sample (see FIGS. 13A-13B).

### Example 18

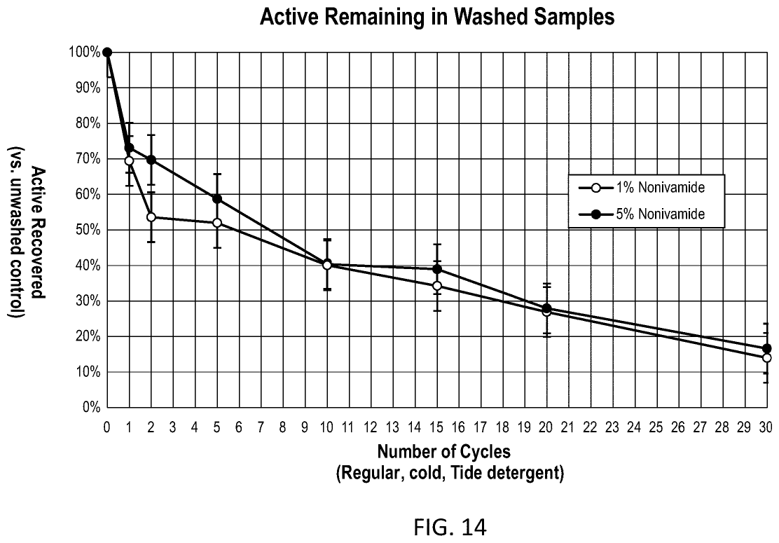

Samples were produced at two different active loadings (1% and 5% nonivamide. Samples were then washed 1, 2, 5, 15, 20 and 30 times in cold water, with one TIDE® detergent pod, using a regular wash setting (about 40 minutes). Active remaining was quantified and plotted with respect to an unwashed control sample (see FIG. 14).

### Example 19

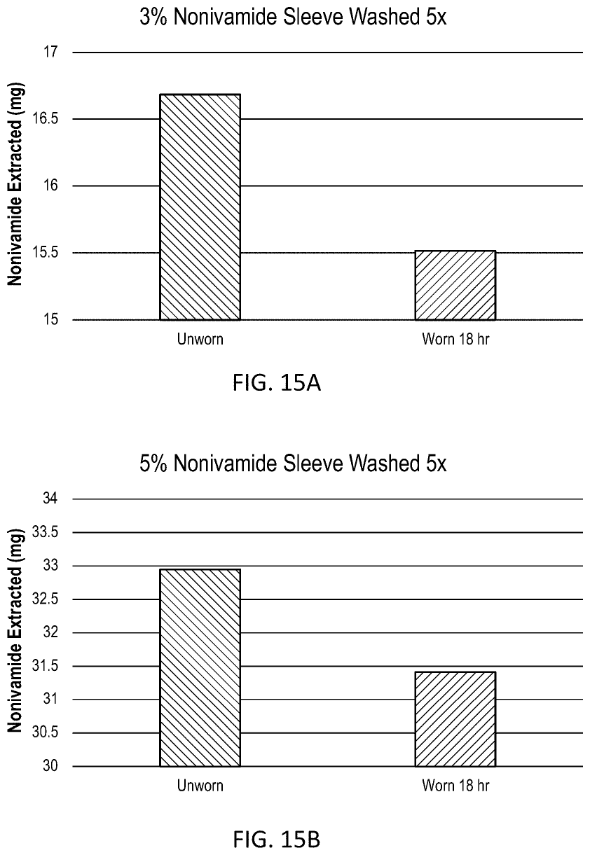

Sleeves made from activated yarn, prepared as disclosed herein, were washed multiple times and worn for extended periods. Solvent extraction determined the mass of nonivamide remaining in the garment and the average rate of nonivamide release over the duration of the wear (see FIGS. 15A-15B). It was found that after several washes, the sleeves released within the effective dose range, and retained enough active for an additional 25-35 doses. Based on a typical topical cream, the effective daily dose of capsaicin for relief in the foot, ankle, and calf range from 0.5 to 6 mg depending on the severity of pain. Experimental results generated by wear testing has shown that after multiple washings, the activated yarn still released active comfortably within this range (1.8 to 2.4 mg). While the majority of active losses are incurred during wash cycles, it has been determined that after 30 washes, a sock made from activated yarn may retain enough active for 10 or more additional doses.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

The invention claimed is:

1. A wearable article comprising:
a fabric formed with at least one yarn, yarn precursor, or filament having a polymer, oligomer, or monomer matrix including at least one active composition applied thereto;
wherein the at least one yarn, yarn precursor, or filament includes at least one occluded area and at least one exposed area, wherein the at least one occluded area restricts release of the at least one active composition, and wherein the at least one exposed area does not restrict release of the at least one active composition; and
wherein the wearable article persistently releases the at least one active composition when in contact with a skin surface of a wearer via the at least one exposed area.

2. The wearable article of claim 1, wherein the wearable article is an ankle sleeve, an arm sleeve, a calf sleeve, a knee sleeve, a lower leg sleeve, a wrist sleeve, a sock, an insole, a glove, tights, leggings, partial leggings, pants, partial pants, a shirt, or a partial shirt.

3. The wearable article of claim 1, wherein the at least one active composition includes a capsaicinoid.

4. The wearable article of claim 3, wherein the capsaicinoid is nonivamide.

5. The wearable article of claim 1, wherein the at least one active composition is persistently released following at least one wash when the wearable article is in contact with the skin surface of the wearer.

6. The wearable article of claim 1, wherein the wearable article is comprised of a knitted fabric formed from the at least one yarn, yarn precursor, or filament.

7. The wearable article of claim 1, wherein a structural integrity and the persistent release of the at least one composition of the wearable article are maintained after at least one wash, and wherein the wearable article is reusable after at least one wash.

8. The wearable article of claim 1, wherein the wearable article is operable to substantially conform to at least one body part of the wearer.

9. The wearable article of claim 1, wherein the wearable article is seamless.

10. The wearable article of claim 1, wherein the wearable article loses less than about 25% of the at least one active composition present in the material during a wash cycle.

11. The wearable article of claim 1, wherein the at least one occluded area comprises at least 80% of a surface area of the at least one yarn, yarn precursor, or filament.

12. The wearable article of claim 1, wherein the fabric further includes an air-covered yarn.

13. The wearable article of claim 1, wherein the wearable article produces a compressive force when worn.

14. The wearable article of claim 1, wherein the at least one occluded area is formed using at least one coating, and wherein the at least one coating is substantially impermeable to the at least one active composition.

15. A wearable article comprising:
a knitted structure formed with at least one substrate having a polymer, oligomer, or monomer matrix including at least one active composition applied thereto;
wherein the knitted structure includes at least one occluded area and at least one exposed area, wherein the at least one occluded area restricts release of the at least one active composition, and wherein the at least one exposed area does not restrict release of the at least one active composition;
wherein the knitted structure is operable to substantially conform to at least one body part of a wearer; and
wherein the wearable article persistently releases the at least one active composition when in contact with a skin surface of a wearer via the at least one exposed area.

16. The wearable article of claim 15, wherein the wearable article is an ankle sleeve, an arm sleeve, a calf sleeve, a knee sleeve, a lower leg sleeve, a wrist sleeve, a sock, an insole, a glove, tights, leggings, partial leggings, pants, partial pants, a shirt, or a partial shirt.

17. The wearable article of claim 15, wherein the knitted structure is formed with a first substrate and a second substrate, wherein the first substrate includes the at least one active composition, and wherein the second substrate does not include an active composition.

18. The wearable article of claim 15, wherein the at least one active composition includes a first active composition and a second active composition, wherein the at least one substrate includes a first substrate and a second substrate, wherein the first substrate includes the first active composition, and wherein the second substrate includes the second active composition.

19. The wearable article of claim 15, wherein the at least one substrate is a yarn, a yarn precursor, a thread, a fiber, a filament, or a textile.

20. A wearable article comprising:
a fabric formed with at least one substrate having a polymer, oligomer, or monomer matrix including at least one active composition applied thereto;
wherein the at least one substrate includes at least one occluded area and at least one exposed area, wherein the at least one occluded area restricts release of the at least one active composition, and wherein the at least one exposed area does not restrict release of the at least one active composition;
wherein the wearable article is operable to substantially conform to at least one body part of a wearer;
wherein the at least one body part is a finger, a hand, a wrist, an elbow, an arm, a shoulder, a torso, a head, a face, a neck, a toe, a foot, an ankle, a knee, a hip, and/or a leg; and
wherein the wearable article persistently releases the at least one active composition when in contact with a skin surface of a wearer via the at least one exposed area.
